(19) United States
(12) Patent Application Publication
(10) Pub. No.: US 2016/0340731 A1
(43) Pub. Date: Nov. 24, 2016

(54) METHOD

(71) Applicants: IMPERIAL INNOVATIONS LTD, London (GB); UNIVERSITE DU QUEBEC A CHICOUTIMI, Chicoutimi (CA); PRESIDENT AND FELLOWS OF HARVARD COLLEGE, Cambridge, MA (US); SWANSEA UNIVERSITY, Swansea (GB); THE ROYAL INSTITUTION FOR THE ADVANCEMENT OF LEARNING/MCGILL UNIVERSITY, Montreal (CA)

(72) Inventors: William O.C.M. COOKSON, London (GB); Miriam F. MOFFATT, London (GB); Catherine LAPRISE, Chicoutimi (CA); Mark LATHROP, Montreal (CA); Liming LIANG, Boston, MA (US); Gwyneth DAVIES, Swansea (GB)

(21) Appl. No.: 15/114,388

(22) PCT Filed: Jan. 27, 2015
(86) PCT No.: PCT/EP2015/051622

§ 371 Date: Jul. 26, 2016

(30) Foreign Application Priority Data
Jan. 27, 2014 (GB) 1401385.8
Dec. 31, 2014 (GB) 1423387.8

(51) Int. Cl. C12Q 1/68

(52) U.S. Cl. CPC C12Q 1/6883; C12Q 2600/154

(57) ABSTRACT

A method of identifying a subject falling within a new patient population characterised by eosinophil IgE mediated allergic inflammation, involving analysing the level of methylation in a DNA sample obtained from the subject for one or more promoter regions associated with one or more genes. Individuals within this new patient population are expected to be likely to respond to therapies for eosinophil IgE mediated inflammation, such as inhibitors of IL-5, IL-13, IgE or M1 prime activity and other therapies directed towards eosinophils.

The present disclosure relates to a method of identifying a human subject with eosinophil IgE mediated allergic inflammation, and optionally subsequent treatment of the same. The disclosure is based on the identification of a new patient population, which is characterised by epigenetic changes in CpG islands (CGI) of their DNA.

### BACKGROUND

There are about 52 million people worldwide that suffer from asthma and about 1.4 million of these fall into the category of severe asthmatics. Asthma accounts for one-quarter of all emergency room visits in the US each year, about 1.75 million, and more than 10 million outpatient visits and 479,000 hospitalizations, according to statistics from the Asthma and Allergy Foundation of America. The estimated cost of asthma in the US alone is $18 billion, of which $10 billion are direct costs from events like hospitalizations, the foundation says. It has been suggested that in the UK, 1.1 million working days are lost due to breathing or lung problems associated with asthma. In addition, the number of cases of asthma seems to increasing. One estimate is that by the year 2025 there will be 100 million asthma sufferers.

A significant proportion of patients diagnosed with asthma are children. It is estimated that one in about eleven children in the US have asthma. The figures in the UK suggest that one child is admitted to hospital every 18 minutes because of asthma.

Acute attacks of asthma are relatively dangerous and if the patient does not get access to proper medical assistance quickly then the patient can die. In 2010, in the UK, there were 1,143 deaths from asthma, 16 of which were children under 14 years of age. In 2007, in the US, asthma was linked to 3,447 deaths (about 9 per day). One figure from the American Academy of Allergy, Asthma & Immunology is that there are 250,000 asthma-associated deaths each year worldwide and that almost all of those deaths could be avoided with better long term medical treatment.

The basic treatment for these indications is steroids (glucocorticoids) which reduce the severity of the allergic response. In the case of asthma and rhinitis, the steroids are usually inhaled and may be provided as a combination therapy with a beta-agonist, such as a long acting beta agonist. Examples of this kind of therapy include the combination product Advair (combination of the steroid Fluticasone and beta-agonist Salmeterol), which had worldwide sales of approximately $7.72 billion US dollars in 2012.

However, there are some patients who can be categorised as severe because their condition is not well controlled by the currently available therapies.

Drug companies have responded to this unmet medical need by developing biological therapeutics for the treatment of severe asthma. These new therapies include antibodies which inhibit IL-5 activity, IL-13 activity, IgE (Xolair) or M1 prime activity. AstraZeneca, GlaxoSmithKline, Teva and Roche/Genentech all have biologics in the clinic or approved for asthma.

However, these therapies are likely to be relatively expensive, for example in the region of $15,000 to $25,000 US dollars per year per patient.

It is therefore necessary to robustly identify those patients who can benefit from these new therapies, to minimise the impact on healthcare budgets.

### Disease Pathology and Intervention

In this respect, the diseases of asthma, eczema (atopic dermatitis) and hay fever (allergic rhinitis) are typified by Immunoglobulin E (IgE) mediated reactions to common allergens. Atopic mechanisms contribute strongly to symptoms and manifestations of these diseases. Immunoglobulin E acts as the central mediator of the atopic state through binding to high and low affinity receptors, and therapies directed against IgE are of benefit not only to patients with asthma,^{1,2} but also to patients with allergic rhinitis² and atopic dermatitis.³ Atopic inflammation has been intensively studied,^{4,5} leading to the recognition that IgE creation in B-cells is promoted by the presence of Interleukin-4 (IL-4) and IL-13 released from T helper cell type 2 (TH2) cells and eosinophils.^{6,7} Eosinophils contain many potent pro-inflammatory molecules and are major effectors of atopic inflammation.

Therapies for atopic diseases may be directed against IgE itself (for example the antibody therapy omalizumab), or against T-cell responses to allergens (for example with immunotherapy), or against eosinophils or cytokines (such as IL-5) and their receptors that support eosinophil proliferation or infiltration.

Thus, patients who have high total IgE serum levels may especially benefit from therapies which control IgE levels. However, circulating IgE only partially reflects inflammatory events that are taking place in the airways and skin. For example, it has been shown that eosinophils directly regulate IgE production by local actions in the bone-marrow.⁴⁵

In practical terms, total serum IgE has been of limited use in predicting the outcome of therapies for atopic diseases. Whilst a method of identifying patients who will respond to therapies directed against IgE or eosinophils would be useful, to date no robust method has been identified. Instead, current diagnostic methods rely mostly on the physicians' clinical observations.

Part of the reason for the lack of a satisfactory method for identifying this patient population is because the knowledge of genes controlling IgE production is incomplete. For instance, genome-wide association studies have consistently shown polymorphisms in STAT6, the high-affinity receptor for IgE (FCERIA), the IL4/RAD50 locus and several HLA genes within the MHC to be associated with high levels of the total serum IgE concentration⁸⁻¹⁰.

However, SNPs in these genes in combination account for only 1-2% of the total variation in total serum IgE⁹. Furthermore, these studies have not identified novel pathways or potential new therapeutic targets. This suggests that there are other as yet unidentified genes which may account for the remainder of the total variation in total serum IgE levels.

A promising approach to gene identification relies on the genome-wide examination of epigenetic changes in the regulatory regions of genes. CpG methylation is associated with gene silencing and the patterns of gene expression that determines cellular types and functions¹¹. Islands of CpG (CGI) sequences are positioned in or near the promoters of 40% of human genes¹². Abnormalities of DNA methylation are well recognised in single gene disorders and in cancer¹³. It is expected that epigenetic changes in methylation will be of importance to the understanding of common human diseases¹³.

It has previously been established that IL-4 expression is related to upstream epigenetic variation in DNA methylation in T-cells,¹⁴. Other murine in vivo studies investigating methylation of IFN-gamma and IL-4 promoters concluded there was no correlation between gene methylation and IgE, which suggested that methylation of CpG sites did not regulate directly the IgE response in mice. In addition they concluded that the biological significance of the epigenetic changes they observed was uncertain⁴⁸.

Despite these previous studies which suggest that epigenetic changes may not be important in the regulation of IgE, the present inventors have nonetheless searched systematically for epigenetic factors associated with IgE serum concentrations in families ascertained through an asthmatic proband, using a robust technology that assays methylation status at single CpG nucleotides within selected CGI across the genome.

### SUMMARY OF THE DISCLOSURE

Interestingly, the present inventors believe that they have identified epigenetic changes in about 33 genes that appear to have a significant biological impact on IgE levels and/or activation. In particular, the results of the work disclosed herein suggest that the methylation patterns in eosinophils are particularly important in the regulation of IgE.

Thus, the present disclosure provides a method of identifying a subject (for example a human subject) falling within a patient population characterised by the presence of eosinophil IgE mediated allergic inflammation comprising the steps of:
a. analysing a DNA sample obtained from the subject for the level of methylation in one or more promoter regions associated with one or more genes selected from the group consisting of LPCAT2, IL5RA, ZNF22, L2HGDH, IL4, SLC25A33, RB1, SERPINC1, TFF1, SKC17A4, L2HGDH, TMEM86B, COL15A1, CEL, SPINK4, ADARB1, SEPT12, TMEM52B, FAM112A, SLC7A11, KEL, PIK3CB, TMEM41A, PDE6H, KLF1, ITAG2B, PRG3, SLMAP, PRG2, EFNA3, SLC43A3, CLC, ALDH3B2, GATA1, CCR3 and IL1RL1; and
b. assigning the subject as a member of the patient population with eosinophil IgE mediated allergic inflammation where there is low methylation in one or more of the promoter regions.

Advantageously, the method is robust and independent of many factors such as age, sex, smoking and the like.

### BRIEF DESCRIPTION OF THE FIGURES

FIG. 1 Manhattan plot of the results of the genome-wide methylation association study

The results of genome-wide association testing to CGI are shown for the MRCA panel of families. The horizontal line illustrates the threshold for a False Discovery Rate (FDR) <0.01.

FIG. 2 Scatter plot showing association of selected CpG loci to total serum IgE concentrations in the MRCA panel, partitioned by eosinophil counts

Methylation on the abscissa (x) is normalised around a mean of 0. Black dots indicate subjects with eosinophil counts greater than the median for the MRCA panel.

FIG. 3 Boxplots of methylation at selected CGI in isolated eosinophils from subjects with and without asthma and high total serum IgE concentrations (>110 IU/I)

Methylation (β) is shown on a scale of 0-1. The intensity of the data point colour is proportion to total serum IgE. All CGI exhibited reduced variability and levels of methylation in the subjects with asthma and high IgE (P<0.05).

FIG. 4 Graph showing concordance in methylation status at IgE-associated loci when comparing whole-genome bisulphite sequencing (WGBS) with the Illumina platform

The graphs show a comparison between IgE-associated CpG probes using Illumina 450K (x-axis) and WGBS (y-axis) platforms for two samples (1 and 2) with 20-fold sequence coverage.

FIGS. 5A & 5B Boxplots showing distribution of methylation status at IgE-associated loci in isolated leukocyte subsets

The distribution of methylation in peripheral blood leukocyte subsets at the most strongly IgE-associated loci is shown. CpG methylation was measured by Illumina Infinium 450K platform. Boxplots show means and interquartile ranges (a), (c), (e), (g), (l) and (k). Results from publically available data was derived from 6 healthy controls¹⁶.

Lower levels of methylation with wider variation is observed in eosinophils when compared to whole blood (WB) and subsets comprising CD14+ Monocytes (CD14+M); CD19+B cells (CD19+B); CD4+T-cells (CD4+T); CD56+ natural killer cells (CD56+NK); CD8+T cells (CD8+T); granulocytes (Gran); Neutrophils (Neu) and PBMC (b), (d), (f), (h), (j), and (l).

Eosinophils (Eos) from 24 subjects in the SLSJ panel also showed lower levels of methylation with wider variation compared to whole blood (WB, 22 SLSJ subjects) and to subsets including B-cells (BC, 9 control subjects), Monocytes (Mono, 76 control subjects), and T-cells (TC, 74 control subjects).

FIG. 6 Graph showing power estimations to detect eosinophil-specific effects in DNA from peripheral blood lymphocytes

The graph shows that the original MRCA dataset (grey line) and the combined dataset (black line) are well powered to detect signals of the magnitude observed in the three groups of subjects. The horizontal line shows the power of sample size of 6 described in Reinius et al¹⁶ to detect differences in CpG metylation in unfractionated PBL. The mean variance (as standard deviation, SD) for the IgE-associated loci was 0.036 in PBLs from the primary MRCA panel and 0.023 in the whole blood normal samples from Reinius et al¹⁶, demonstrating that the results obtained were consistent with the previous experiment performed by Reinius et al.

### DETAILED DESCRIPTION OF THE DISCLOSURE

In one embodiment, the low methylation is defined as a level of methylation that is at least 2 standard deviations less than the mean level of methylation in the same one or more promoter regions in a control sample.

A “control sample” as employed herein refers to a sample obtained from an individual who does not have eosinophil IgE mediated allergic inflammation.

In another embodiment, the low methylation is defined as a level of methylation that is below the level of methylation for the 95th percentile of the control population.

In one embodiment, there is low methylation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 of the genes described herein.

In one embodiment, the one or more promoter regions are associated with one or more of the following genes: LPCAT2, IL5RA, ZNF22, L2HGDH, IL4, SLC25A33, RB1, SERPINC1, TFF1, SKC17A4, L2HGDH, TMEM86B, COL15A1, CEL, SPINK4, ADARB1, SEPT12, TMEM52B, FAM112A, SLC7A11, KEL, PIK3CB, TMEM41A, PDE6H, KLF1, ITAG2B, PRG3, SLMAP, PRG2, EFNA3, SLC43A3, CLC, ALDH3B2, GATA1, CCR3 and IL1RL1.

In one embodiment, a promoter region for each of the 36 genes described herein is evaluated.

In one embodiment, only 1, 2, 3, 4, 5, 6, 7, 8, 9, 10 of the genes are evaluated, for example 2 to 5, such as 3 or 4.

In one embodiment the low methylation is associated with cg01998785 adjacent to LPCAT2 (also known as AYTL1). LPCAT2 encodes lyso-platelet-activating factor (PAF) acetyltransferase, which is essential to induce formation of PAF, a potent pro-inflammatory lipid mediator.

In one embodiment, the one or more promoter regions are associated with LPCAT2 and one or more genes selected from the group consisting of IL5RA, ZNF22, L2HGDH, IL4, SLC25A33, RB1, SERPINC1, TFF1, SKC17A4, L2HGDH, TMEM86B, COL15A1, CEL, SPINK4, ADARB1, SEPT12, TMEM52B, FAM112A, SLC7A11, KEL, PIK3CB, TMEM41A, PDE6H, KLF1, ITAG2B, PRG3, SLMAP, PRG2, EFNA3, SLC43A3, CLC, ALDH3B2, GATA1, CCR3 and IL1RL1.

In one embodiment, the genes are selected from the group consisting of SLC25A33, LPCAT2, L2HGDH and a combination thereof.

In one embodiment, the promoter regions are associated with the combination of all three genes SLC25A33, LPCAT2 and L2HGDH.

As discussed above, SNP variations in other genes previously associated with IgE production represents less than 2%, such as 1% or less of the variation in total serum IgE levels and therefore can only identify a very small percentage of the total patient population for a given disease, which is associated with eosinophil IgE mediated allergic inflammation.

In contrast, the percentage of patients with the profile as described herein, for example employing the top three genes SLC25A33, LPCAT2, L2HGDH, represents about 13.5% of the variation in total serum levels and can therefore identify a significantly larger percentage of the total patient population with eosinophil IgE mediated allergic inflammation.

The present method facilitates the identification of those subjects with moderate to severe disease, in particular those whose symptoms are not well controlled by medication. In one embodiment the patient population identified by the present method are those patients with refractory asthma. Refractory or severe asthma as employed herein refers to those patients whose symptoms are not well controlled by inhaled steroids and/or beta2-agonists.

In addition to the top three genes, similar estimates of variance have also been obtained employing other genes identified herein. This makes the method clinically relevant and of practical benefit because it provides for the first time a robust method for identifying patients who would benefit from an alternative therapy.

In one embodiment, the one or more promoter regions are associated with a gene selected from the group consisting of TMEM86B, CEL, CLC and a combination thereof.

In one embodiment, the one or more promoter regions are associated with a gene selected from the group consisting of ZNF22, RB1, KLF and a combination thereof.

In one embodiment, the one or more promoter regions are associated with a gene selected from the group consisting of PRG3, SERPINC1, TFF1, SPINK4 and a combination thereof.

In one embodiment, the eosinophilic IgE mediated inflammation is manifest in the subject as asthma, rhinitis, seasonal rhinitis, atopic dermatitis, anaphylaxis or a combination thereof, such as atopic asthma.

In one embodiment, the patient population is further characterised by high serum IgE levels.

The present inventors have identified variably methylated CpG islands (CGI) with strong and reproducible associations to the total serum IgE concentration. Advantageously, the robustly reproducible CGI associations account for a substantial proportion of variation in total serum IgE that is 10-fold higher than that derived from other large SNP genome wide association studies.

The independent associations of these CGI to eosinophil counts suggest that the CGI methylation may have captured gene regulation events taking place in eosinophils. The ex vivo examination of the principal CGI loci in eosinophils isolated from asthmatics and controls gives results that are consistent with the process of eosinophil activation³⁴ and with the suspected mixture of activated and unactivated eosinophil populations in human blood³³.

Clinically, the presence of eosinophilia in the peripheral blood or airways identifies a subgroup of refractory asthmatics³⁵ and therapies directed at eosinophils are effective in some of these patients³⁶. However, the airways are difficult to access for diagnostic testing (for example the airways in asthma). Furthermore, eosinophilia at the site of disease is poorly reflected by peripheral blood eosinophil counts (Ullmann N, Bossley C J, Fleming L, Silvestri M, Bush A, Saglani S. Blood eosinophil counts rarely reflect airway eosinophilia in children with severe asthma. Allergy. 2013 March; 68(3):402-6. doi: 10.1111/all.12101. Epub 2013 Jan. 25). Thus, in many instances clinicians are left to a process of trial and error to establish if the patient is a severe asthmatic that will respond to alternative therapy.

Advantageously, the methylation status at the loci described above may be used to identify patients most likely to respond to therapies directed against eosinophils or individual gene products.

Cigarette smoking may increase serum IgE. An anti-correlated associations to current cigarette smoking was observed with F2RL3 (P=8.6×10−¹⁷) and GPR15 (P=4.6×10−⁹). The SLSJ dataset confirmed these associations (P=2.5×10−⁶ and P=6.6×10−⁷). Thus in one embodiment the present method is adjusted for these smoking related genes. Having said that adjusting for smoking had minimal impact on the top hits for IgE and neither locus affected IgE in our subjects.

In one embodiment, the method comprises a further step of administering an anti-inflammatory therapy to a subject assigned as a member of the patient population characterised by eosinophil IgE mediated inflammation.

In one embodiment, the subject is a mammal, for example a human.

In one embodiment, the method herein further comprises the step of administering an alternative anti-inflammatory therapy to a subject assigned as a member of a patient population characterised by eosinophil IgE mediated inflammation.

In one embodiment, the method comprises a further step of administering to a subject assigned as a member of a the patient population with eosinophil IgE mediated inflammation a therapeutically effective amount of a biological medication, for example for said eosinophil IgE mediated inflammation, such as an inhibitor of IL-5 activity, IL-13 activity, IgE or M1 prime activity.

An “inhibitor against IL-5 action/activity” as employed herein refers to an agent that blocks or prevents signalling, activation/stimulation through the interaction of IL-5 and its corresponding receptor. In one embodiment the inhibitor is directed at IL-5. In one embodiment the inhibitor is directed against the IL-5 receptor. Examples of inhibitors of IL-5 activity include Benralizumab, Mepolizumab and Reslizumab.

An “inhibitor against IL-13 action/activity” as employed herein refers to an agent that blocks or prevents signalling, activation/stimulation through the interaction of IL-13 and its corresponding receptor. In one embodiment the inhibitor is directed at IL-13. In one embodiment the inhibitor is directed to the IL-13 receptor. Examples of inhibitors of IL-13 activity include an inhibitor selected from Tralokinumab and Lebrikizumab.

An “inhibitor against IgE action/activity” as employed herein refers to an agent that blocks or prevents activation/production of IgE, for example by binding to IgE itself or by inhibiting one or more proteins involved in IgE production. An example of an IgE inhibitor is Omalizumab.

An “inhibitor against M1 prime action/activity” as employed herein refers to an agent that blocks or prevents signalling, activation/stimulation through the interaction of M1 prime and its corresponding receptor. In one embodiment the inhibitor is directed at M1 prime. In one embodiment the inhibitor is directed against the M1 prime receptor. An example of an M1 prime inhibitor is Quilizumab.

“Biological medication” as employed herein refers to medication based on proteins, peptides, DNA, RNA, gene therapy or similar technology. The term is used interchangeably with “biological therapeutics”.

In one embodiment, the therapy is provided in combination with a known therapy, such as steroid therapy, such as inhaled steroids or combinations of inhaled steroids, beta2 agonists, for example long acting beta2 agonist, pI3 kinase inhibitors, for example as disclosed in WO2011/04811 and p38 kinase inhibitors such as those disclosed in WO2010/038086.

In one embodiment, the known therapy is a therapy directed towards eosinophils. These include for example, the anti-IL-5 antibodies, Mepolizumab (GlaxoSmithKline, Research Triangle Park, NC) and Reslizumab (SCH55700, Cinquil; Teva Pharmaceuticals, Petah Tikva, Israel); and the anti-CD52 antibody, Alemtuzumab.

In one embodiment, the alternative therapy is a biological therapeutic agent, for example an antibody or binding fragment thereof.

In one embodiment, the method comprises a first step of determining the serum IgE levels in the subject.

In one embodiment, the method does not comprise a first step of determining the serum IgE levels in the subject and the serum IgE levels may analysed as part of the method disclosed herein.

In one embodiment, the method comprises a first step of determining if the subject suffers from an allergic inflammatory condition, such as asthma rhinitis, seasonal rhinitis, atopic dermatitis, anaphylaxis or a combination thereof, such as atopic asthma.

In one embodiment, the DNA sample for analysis in the method according to the disclosure is obtained from eosinophils.

Advantageously, because the required DNA sample can be obtained from a blood sample, the present method provides a systemic test for inflammatory events which operate locally in tissues. This obviates the requirement to obtain a tissue sample from the relevant site of inflammation, which in many instances can be difficult and invasive.

The present disclosure relates to a new patient population characterised by high IgE serum levels and allergic inflammatory responses activated by eosinophils triggering IgE responses. This patient population have conditions such as atopic asthma, rhinitis, atopic dermatitis, food allergies and the like. Thus in one embodiment there is provided a method of treating a patient identified with low methylation in one or more genes identified herein and optionally with high IgE levels, for inflammation, such as eosinophil and/or IgE mediated inflammation, in particular asthma, rhinitis, dermatitis, food allergies or similar.

“Eosinophil and IgE mediated allergic inflammation” or “eosinophil mediated inflammation” are used interchangeably herein and refers to an inflammatory response which is modified by IgE or the presence of activated eosinophils or both, wherein the production of IgE and the release of damaging inflammatory mediators is enhanced by eosinophil activation.

A tissue sample or peripheral blood sample may be obtained from the human subject, by known techniques. In one embodiment, the sample for use in the method is a blood sample.

In one embodiment, the method according to the present disclosure does not include the step of obtaining the blood sample or tissue sample.

Total serum IgE levels and specific serum IgE levels can be measured using the Immunocap FEIA (Pharmacia AB, Uppsala, Sweden) or an equivalent assay.

In one embodiment, the method comprises the step of contact the DNA from a patient tissue or blood sample with a material(s) employed in a DNA methylation assay. Assays for establishing DNA methylation include methylation specific PCR, whole genome bisulfite sequencing, HELP assays (based on restriction enzyme specificity to methylated/unmethylated CpG sites) ChIP-on-chip assays, restriction landmark genomic scanning, methylated DNA immune precipitation, pyrosequencing, and methylCpG binding proteins.

In one embodiment the level of methylation is determined using a method selected from the group consisting of bisulphite sequencing, microarrays and bead arrays, in particular 450K bead arrays (IIlumina Inc, San Diego, Calif., USA)

The subjects' DNA may be extracted from the sample, for example by employing phenol-chloroform after red cell lysis and centrifugation to recover leukocyte nuclear pellets. DNA samples may be bisulfite converted using the Zymo EZ DNA Methylation kit (Zymo Research, Orange, Calif., USA).

In one embodiment, about 1000 ng of input DNA is employed in the methylation analysis.

DNA can be extracted from blood cells, for example from eosinophils employing the QIAamp® DNA Blood Mini Kit.

Isolation of eosinophils is as described in the art^{23,24}, incorporated herein by reference.

“Reagents” as employed herein refer to chemical reagents, in liquid or solid form employed in the analysis.

“Materials” as employed herein refers to chips employed in the analysis or other materials like beads, etc.

The methylation analysis is performed in accordance with the instructions supplied with the Illumina Infinium kit, and employing for example the HumanMethylation27 BeadChips (Illumina Inc, San Diego, Calif., USA).

These materials and reagents interrogate 27,578 CpG sites for the extent of DNA methylation.

Data can be visualized using the BeadStudio software (Illumina Inc, San Diego, Calif., USA). Samples that fail quality control should generally be repeated.

Signal intensities of methylated (Signal B) and unmethylated probes (Signal A) can be exported from the BeadStudio interface, along with detection P-values representing the likelihood of detection relative to background.

Individual data points with P values outside the detection criteria (P>0.05) may be treated as missing data.

Methylation can also be assessed using Illumina 450K arrays, or direct assays based on bisulphate sequencing, or antibody assays. Other methods of analysing levels of methylation will be known to the skilled person and the methods disclosed in the present disclosure are not limiting.

36 genes were identified where the promoter regions associated therewith have low levels of methylation. Said genes are listed above.

“Eosinophil activation” as employed herein refers to activation by various means, including cross-linking of IgG or IgA Fc receptors with IgG, IgA, or secretory IgA, with the latter being most potent. Eosinophils can also be primed for activation by a number of mediators, including IL-3, IL-5, GM-CSF, CC chemokines, and platelet-activating factor.

Upon activation, eosinophils produce various immune effector molecules such as cationic granule proteins (e.g. major basic protein and eosinophil cationic protein), reactive oxygen species such as peroxide, cytokines such as IL-1, IL-5, IL-13 and TNF alpha, and can also enhance IgE production. Accordingly, eosinophils are an important mediator of the allergic response.

“Associated promoter region” or “promoter region” as employed herein refers to the genomic region upstream of a given gene (towards the 5′ region), which includes the core promoter and proximal promoter together with the primary regulatory elements. The precise location and size of the promoter region varies between genes but typically encompasses the genomic region from about 250 base pairs upstream of the gene to the transcription start site.

Specifically, the promoter region for each of the loci discovered can be identified by the chromosome on which the target locus is located; the genomic position of C in CG dinucleotide (for a particular database source and version; the transcription start site genomic coordinate; the gene strand (i.e. either positive or negative); the RefSeq gene identifier (GeneID); the Gene Symbol; the Gene synonyms; the Gene Accession number (this is the accession of the longest transcript); the GI ID; the gene annotation from NCBI database; the gene product description from NCBI database; the distance of CG dinucleotide to transcription start site; a Boolean true/false variable denoting whether the loci is located in a CpG island; the chromosomal location and genomic coordinates of the CpG island from NCBI database; the chromosome:start-end of upstream CPG island from a micro RNA; and the Name of micro RNA near the locus (see Table 3).

In the method disclosed herein, a promoter region is associated with one gene, so where there are multiple genes employed in the method, there will be a corresponding number of promoters; for example 3 different promoter regions corresponding to 3 different genes or 2 different promoter regions corresponding to 2 different genes. There may also be more than one promoter region evaluated per gene; for example 2 different promoter regions, both of which are associated with the same gene.

The new patient population defined herein is characterised by high levels of IgE in serum and low levels of methylation in a promoter associated with a gene listed herein.

“High levels” of serum IgE as employed herein refers to an elevation of the total serum IgE beyond the 20^{th} percentile, such as beyond the 10^{th} percentile of the age and sex adjusted normal distribution for a given population.

“Low levels” of methylation in the relevant promoter as employed herein refers to levels below the 10^{th} percentile such as below the 5^{th} percentile of the distribution (or two standard deviations of the mean) in the normal population. The threshold for “low levels” of methylation will vary depending on the promoter region and the methylation assay used.

“Biological therapeutics” as employed herein refer to agents prepared using recombinant techniques, as opposed to agents which are chemically synthesised, for example based on such as proteins, viruses, DNA or similar.

In one embodiment, the biological therapeutic is an antibody or a binding fragment thereof, for example a neutralising antibody.

In one embodiment, the biological therapeutic is delivered parenterally.

In one embodiment, the biological therapeutic such as an antibody is delivered by inhalation therapy, in particular an IL-14 antibody or binding fragment thereof.

Monoclonal antibodies may be prepared by any method known in the art such as the hybridoma technique (Kohler and Milstein, 1975, Nature, 256:495-497), the trioma technique, the human B-cell hybridoma technique (Kozbor et al, 1983, Immunology Today, 4:72) and the EBV-hybridoma technique (Cole et al, Monoclonal Antibodies and Cancer Therapy, p77-96, Alan R Liss, Inc., 1985).

Antibodies for use in the invention may also be generated using single lymphocyte antibody methods by cloning and expressing immunoglobulin variable region cDNAs generated from single lymphocytes selected for the production of specific antibodies by for example the methods described by Babcook, J et al, 1996, Proc. Natl. Acad. Sci. USA 93(15):7843-7848, WO92/02551 and WO02004/051268 and WO2004/106377.

An “antigen-specific antibody” as employed herein is intended to refer to an antibody that only recognises the antigen to which it is specific or an antibody that has significantly higher binding affinity to the antigen to which it is specific compared to binding to antigens to which it is non-specific, for example at least 5, 6, 7, 8, 9, 10 times higher binding affinity.

Chimeric antibodies are those antibodies encoded by immunoglobulin genes that have been genetically engineered so that the light and heavy chain genes are composed of immunoglobulin gene segments belonging to different species. Bivalent antibodies may be made by methods known in the art (Milstein et al, 1983, Nature 305:537-539; WO93/08829, Traunecker et al, 1991, EMBO J. 10:3655-3659). Multi-valent antibodies may comprise multiple specificities or may be monospecific (see for example WO92/22853).

In one embodiment, the antibody for use in the present invention is humanised. As used herein, the term ‘humanised antibody molecule’ refers to an antibody molecule wherein the heavy and/or light chain contains one or more CDRs (including, if desired, one or more modified CDRs) from a donor antibody (e.g. a murine monoclonal antibody) grafted into a heavy and/or light chain variable region framework of an acceptor antibody (e.g. a human antibody) (see, e.g. U.S. Pat. No. 5,585,089; WO91/09967). For a review, see Vaughan et al, Nature Biotechnology, 16, 535-539, 1998.

In one embodiment, rather than the entire CDR being transferred, only one or more of the specificity determining residues from any one of the CDRs described herein above are transferred to the human antibody framework (see for example, Kashmiri et al., 2005, Methods, 36, 25-34). In one embodiment only the specificity determining residues from one or more of the CDRs described herein above are transferred to the human antibody framework. In another embodiment only the specificity determining residues from each of the CDRs described herein above are transferred to the human antibody framework.

In a humanised antibody of the present invention, the framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

When the CDRs or specificity determining residues are grafted, any appropriate acceptor variable region framework sequence may be used having regard to the class/type of the donor antibody from which the CDRs are derived, including mouse, primate and human framework regions.

Suitably, the humanised antibody has a variable domain comprising human acceptor framework regions as well as one or more of CDRs. Thus, provided in one embodiment is a humanised antibody which binds human IL-5, IL-13, M1 prime or IgE wherein the variable domain comprises human acceptor framework regions and non-human donor CDRs.

Examples of human frameworks which can be used in the present invention are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/

In a humanised antibody of the present invention, the acceptor heavy and light chains do not necessarily need to be derived from the same antibody and may, if desired, comprise composite chains having framework regions derived from different chains.

The antibodies for use in the present invention can also be generated using various phage display methods known in the art and include those disclosed by Brinkman et al. (in J. Immunol. Methods, 1995, 182: 41-50), Ames et al (J. Immunol. Methods, 1995, 184:177-186), Kettleborough et al (Eur. J. Immunol. 1994, 24:952-958), Persic et al. (Gene, 1997 187 9-18), Burton et al (Advances in Immunology, 1994, 57:191-280) and WO 90/02809; WO91/10737; WO92/01047; WO92/18619; WO93/11236; WO95/15982; WO95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. Techniques for the production of single chain antibodies, such as those described in U.S. Pat. No. 4,946,778 can also be adapted to produce single chain antibodies to IL-5, IL-13, M1 prime or IgE polypeptides. Also, transgenic mice, or other organisms, including other mammals, may be used to express humanised antibodies.

The antibody used in the present invention may comprise a complete antibody molecule having full length heavy and light chains or a fragment thereof and may be, but are not limited to Fab, modified Fab, Fab′, modified Fab′, F(ab′)2, Fv, single domain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech 23(9):1126-1136; Adair and Lawson, 2005, Drug Design Reviews—Online 2(3), 209-217). The methods for creating and manufacturing these antibody fragments are well known in the art (see for example Verma et al., 1998, Journal of Immunological Methods, 216, 165-181). Other antibody fragments for use in the present invention include the Fab and Fab′ fragments described in WO2005/003169, WO2005/003170 and WO2005/003171. Multi-valent antibodies may comprise multiple specificities e.g bispecific or may be monospecific (see for example WO92/22853, WO05/113605, WO2009/040562 and WO2010/035012).

In one embodiment, the antibody is provided as an IL-5, IL-13, M1 prime or IgE binding antibody fusion protein which comprises an immunoglobulin moiety, for example a Fab or Fab′ fragment, and one or two single domain antibodies (dAb) linked directly or indirectly thereto, for example as described in WO2009/040562, WO2010/035012, WO2011/030107, WO2011/061492 and WO2011/086091, all incorporated herein by reference.

In one embodiment, the fusion protein comprises two domain antibodies, for example as a variable heavy (VH) and variable light (VL) pairing, optionally linked by a disulphide bond.

In one embodiment, the Fab or Fab′ element of the fusion protein has the same or similar specificity to the single domain antibody or antibodies. In one embodiment the Fab or Fab′ has a different specificity to the single domain antibody or antibodies, that is to say the fusion protein is multivalent. In one embodiment a multivalent fusion protein according to the present invention has an albumin binding site, for example a VH/VL pair therein provides an albumin binding site.

Antibody fragments and methods of producing them are well known in the art, see for example Verma et al, 1998, Journal of Immunological Methods, 216, 165-181. Particular examples of antibody fragments for use in the present invention are Fab′ fragments which possess a native or a modified hinge region. A number of modified hinge regions have already been described, for example, in U.S. Pat. No. 5,677,425, WO99/15549, and WO98/25971 and these are incorporated herein by reference.

Further examples of particular antibody fragments for use in the present invention include those described in international patent applications PCT/GB2004/002810, PCT/GB2004/002870 and PCT/GB2004/002871. In particular the modified antibody Fab fragments described in International patent application PCT/GB2004/002810 are provided.

In one embodiment, the antibody heavy chain comprises a CH1 domain and the antibody light chain comprises a CL domain, either kappa or lambda.

In one embodiment, the antibody heavy chain comprises a CH1 domain, a CH2 domain and a CH3 domain and the antibody light chain comprises a CL domain, either kappa or lambda.

The antibody can be of any class (e.g. IgG, IgE, IgM, IgD and IgA) or subclass of immunoglobulin molecule. In one embodiment the antibody for use in the present invention is of IgG class and may be selected from any of the IgG subclasses IgG1, IgG2, IgG3 or IgG4.

The antibody for use in the present invention may include one or more mutations to alter the activity of the antibody. Angal et al (Angal S, King D J, Bodmer M W, Turner A, Lawson A D, Roberts G, Pedley B, and Adair J R 1993. A single amino acid substitution abolishes the heterogeneity of chimeric mouse/human (IgG4) antibody. Mol. Immunol. 30:105-108) describes a site directed mutagenesis approach to minimize half-molecule formation of IgG4 antibodies. In this report, a single amino acid substitution within the core hinge, S241P, resulted in substantially less half-molecule formation. Accordingly, in the embodiment where the antibody is an IgG4 antibody, the antibody may include the mutation S241P. Similar such mutations within the antibodies of the invention are envisaged for the purpose of enhancing the effectiveness and binding efficiency of the antibodies to their target antigens.

It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, R J. Journal of Chromatography 705:129-134, 1995). In one embodiment, the C-terminal lysine of the antibody heavy chain may be absent.

In one embodiment, the medication for said eosinophil IgE mediated inflammation may be in the form of a pharmaceutical composition.

Pharmaceutical compositions maybe conveniently presented in unit dose forms containing a predetermined amount of an active agent of the invention per dose. Such a unit may contain for example but without limitation, 1000 mg/kg to 0.01 mg/kg for example 750 mg/kg to 0.1 mg/kg, such as 100 mg/kg to 1 mg/kg depending on the condition being treated, the route of administration and the age, weight and condition of the subject.

Pharmaceutically acceptable carriers for use in the invention may take a wide variety of forms depending, e.g. on the route of administration.

Compositions for oral administration may be liquid or solid. Oral liquid preparations may be in the form of, for example, aqueous or oily suspensions, solutions, emulsions, syrups or elixirs, or may be presented as a dry product for reconstitution with water or other suitable vehicle before use. Oral liquid preparations may contain suspending agents as known in the art. In the case of oral solid preparations such as powders, capsules and tablets, carriers such as starches, sugars, microcrystalline cellulose, granulating agents, lubricants, binders, disintegrating agents, and the like may be included. Due of their ease of administration, tablets and capsules represent the most advantageous oral dosage unit form in which case solid pharmaceutical carriers are generally employed.

In addition to the common dosage forms set out above, active agents of the invention may also be administered by controlled release means and/or delivery devices. Tablets and capsules may comprise conventional carriers or excipients such as binding agents for example, syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated by standard aqueous or non-aqueous techniques according to methods well known in normal pharmaceutical practice. Pharmaceutical compositions of the present invention suitable for oral administration may be presented as discrete units such as capsules, cachets or tablets, each containing a predetermined amount of the active agent, as a powder or granules, or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active agent with the carrier, which constitutes one or more necessary ingredients. In general, the compositions are prepared by uniformly and intimately admixing the active agent with liquid carriers or finely divided solid carriers or both, and then, if necessary, shaping the product into the desired presentation. For example, a tablet may be prepared by compression or moulding, optionally with one or more accessory ingredients.

Pharmaceutical compositions suitable for parenteral administration may be prepared as solutions or suspensions of the active agents of the invention in water suitably mixed with a surfactant such as hydroxypropylcellulose. Dispersions can also be prepared in glycerol, liquid polyethylene glycols, and mixtures thereof in oils. Under ordinary conditions of storage and use, these preparations contain a preservative to prevent the growth of microorganisms. The pharmaceutical forms suitable for injectable use include aqueous or non-aqueous sterile injection solutions which may contain anti-oxidants, buffers, bacteriostats and solutes which render the composition isotonic with the blood of the intended recipient, and aqueous and non-aqueous sterile suspensions which may include suspending agents and thickening agents. Extemporaneous injection solutions, dispersions and suspensions may be prepared from sterile powders, granules and tablets.

Pharmaceutical compositions can be administered with medical devices known in the art. For example, in a preferred embodiment, a pharmaceutical composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery;

U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollients in creams or ointments and ethanol or oleyl alcohol for lotions. Such carriers may be present as from about 1 percent up to about 98 percent of the composition. More usually they will form up to about 80 percent of the composition. As an illustration only, a cream or ointment is prepared by mixing sufficient quantities of hydrophilic material and water, containing from about 5-10 percent by weight of the compound, in sufficient quantities to produce a cream or ointment having the desired consistency. Pharmaceutical compositions adapted for transdermal administration may be presented as discrete patches intended to remain in intimate contact with the epidermis of the recipient for a prolonged period of time. For example, the active agent may be delivered from the patch by iontophoresis. For applications to external tissues, for example the mouth and skin, the compositions are preferably applied as a topical ointment or cream. When formulated in an ointment, the active agent may be employed with either a paraffinic or a water-miscible ointment base. Alternatively, the active agent may be formulated in a cream with an oil-in-water cream base or a water-in-oil base. Pharmaceutical compositions adapted for topical administration in the mouth include lozenges, pastilles and mouth washes. Pharmaceutical compositions adapted for topical administration to the eye include eye drops wherein the active agent is dissolved or suspended in a suitable carrier, especially an aqueous solvent. They also include topical ointments or creams as above. Pharmaceutical compositions suitable for rectal administration wherein the carrier is a solid are most preferably presented as unit dose suppositories. Suitable carriers include cocoa butter or other glyceride or materials commonly used in the art, and the suppositories may be conveniently formed by admixture of the combination with the softened or melted capier(s) followed by chilling and shaping moulds. They may also be administered as enemas.

In a further aspect, there is provided a kit comprising reagents for use in the method and instructions for use.

In one aspect, there is provided an in vivo animal model for assessing the biological effect of a potential therapeutic agent for eosinophil IgE mediated inflammation comprising dosing the animal model for the said inflammation with the potential therapeutic and then monitoring the level of methylation in a promoter associated with one or more genes selected from the group consisting of LPCAT2, IL5RA, ZNF22, L2HGDH, IL4, SLC25A33, RB1, SERPINC1, TFF1, SKC17A4, L2HGDH, TMEM86B, COL15A1, CEL, SPINK4, ADARB1, SEPT12, TMEM52B, FAM112A, SLC7A11, KEL, PIK3CB, TMEM41A, PDE6H, KLF1, ITAG2B, PRG3, SLMAP, PRG2, EFNA3, SLC43A3, CLC, ALDH3B2, GATA1, CCR3 and IL1RL1, and/or the serum IgE levels.

The animal model may be a mammalian animal model such as a mouse, a rat, a rabbit, a dog, or a primate.

In one embodiment, the animal model is a rodent, for example mouse or rat model.

In one embodiment, the animal for use in the animal model is genetically engineered to facilitate read-out of the animal model.

The disclosure also provides a method of producing an animal model for the purpose of assessing the effect of a test agent on the methylation of one or more genes selected from the group consisting of LPCAT2, IL5RA, ZNF22, L2HGDH, IL4, SLC25A33, RB1, SERPINC1, TFF1, SKC17A4, L2HGDH, TMEM86B, COL15A1, CEL, SPINK4, ADARB1, SEPT12, TMEM52B, FAM112A, SLC7A11, KEL, PIK3CB, TMEM41A, PDE6H, KLF1, ITAG2B, PRG3, SLMAP, PRG2, EFNA3, SLC43A3, CLC, ALDH3B2, GATA1, CCR3 and IL1RL1, and/or the serum IgE levels.

“Comprising” in the context of the present specification is intended to mean “including”.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The invention will now be described with reference to the following examples, which are merely illustrative and should not in any way be construed as limiting the scope of the present invention.

### EXAMPLES

### Subjects

195 siblings and 172 parents were investigated in 95 nuclear pedigrees ascertained through an asthmatic proband, from the MRCA panel in which genome-wide association studies for IgE levels and asthma status^{17,18} were previously carried out. Individual and family histories of respiratory symptoms, demographic information and smoking were assessed at interview using standard questions from the British MRC and ATS questionnaires.^{19,20} ENREF 22 Questionnaires relating to children were administered to a parent, generally the mother.

Replication of the IgE associations was sought in an independent panel of 150 Caucasian subjects selected equally from the top and bottom deciles of IgE distribution in 1614 unselected volunteers (students and staff from Swansea University (PAPA)). Further replication was sought on 160 subjects from a familial asthma collection located in the Saguenay—Lac-Saint-Jean region (SLSJ) from north-eastern Quebec.²¹

### Methods

### Phenotyping

Ethical approval for the study was obtained from the NHS Multicentre Research Ethics Committee for the MRCA subjects; from the Swansea Joint Scientific Research Committee and Swansea Research Ethnics Committee for the Swansea (PAPA) subjects; and from the Le Centre de Santé et des Services Sociaux de Chicoutimi for the SLSJ families. All subjects were submitted to venipuncture at the time of the questionnaire. Differential white cell counts were measured by automated counter. Total serum IgE and specific serum IgE to whole House Dust Mite (Dermatophagoides pteronyssinus) and Timothy grass pollen (Phleum pretense) were measured using the Immunocap FEIA (Pharmacia AB, Uppsala, Sweden). The levels of specific IgE were converted to RAST units according to Pharmacia recommendations. A ‘combined RAST index’ was calculated for each individual as the sum of the RAST scores to HDM and Timothy grass.²²

### Detection of Methylation Status

DNA was extracted by phenol-chloroform after red cell lysis and centrifugation to recover leukocyte nuclear pellets. DNA samples were bisulfite converted using the Zymo EZ DNA Methylation kit (Zymo Research, Orange, Calif., USA) with an input of 1000 ng. The assay was carried out as per the Illumina Infinium Methylation instructions, using the HumanMethylation27 BeadChips (Illumina Inc, San Diego, Calif., USA). These interrogate 27,578 of CpG sites for the extent of DNA methylation. Data were visualized using the BeadStudio software, and samples that failed quality control were repeated. Signal intensities of methylated (Signal B) and unmethylated probes (Signal A) were exported from the from the BeadStudio interface, along with detection P-values representing the likelihood of detection relative to background.

For the Illumina HumanMethylation27 BeadChip data, quantile normalization of intensity was applied to all methylated and unmethylated probes for all samples together. The methylation β values were recalculated as the ratio of methylated prob signal/(total signal+100). The Touleimat and Tost⁵³ analysis pipeline was used for the HumanMethylation450 BeadChip. Individual data points with P outside the detection criteria (P>0.01 or number of beads<3) were treated as missing data, as were samples with more than 20% missing probes. The lumi package⁵⁴ was used for background and colour bias correction. BeadChip ID ad position on chip were included as categorical covariates to account for potential batch effects. Quantile normalization across samples was applied to probes within each functional category (CpG island, shelf, shore, etc.) separately to correct the shift of methylation beta value between Infinium I and Infinium II probes on the Human Methylation450 BeadChip. Probe overlaps with any frequent SNP (MAF >5% in 1000 Genomes Project phase 1 EUR population) in the probe sequence or in position +1 or +2 of the query site (depending on Infinium I or Infinium II status) were removed. Meta-analysis was used to combine the 27K and 450K data with this implementation of the Tost pipeline in order to ensure that the analysis was not confounded by probe differences.

### Isolation of Human Eosinophils

Human eosinophils were isolated as described.^{23,24} Briefly, platelet-rich plasma was removed from 200 ml using centrifugation, followed by Dextran-mediated sedimentation to remove erythrocytes and removal of mononuclear cells using a lymphocyte separation medium. Hypotonic lysis with sterile water removed remaining erythrocytes and other granulocytes were removed using negative selection with anti-CD16 MicroBeads. DNA was extracted using the QIAamp® DNA Blood Mini Kit. Methylation was assessed using IIlumina 450K arrays, with analysis restricted to significantly associated probes from the meta-analysis.

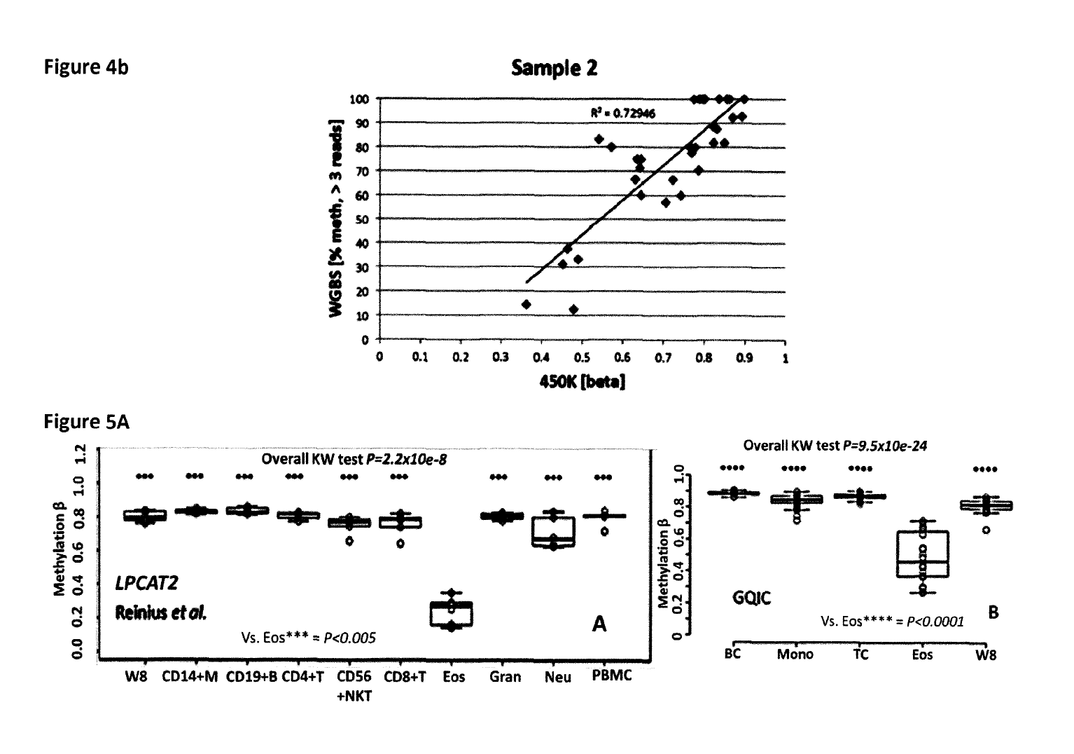

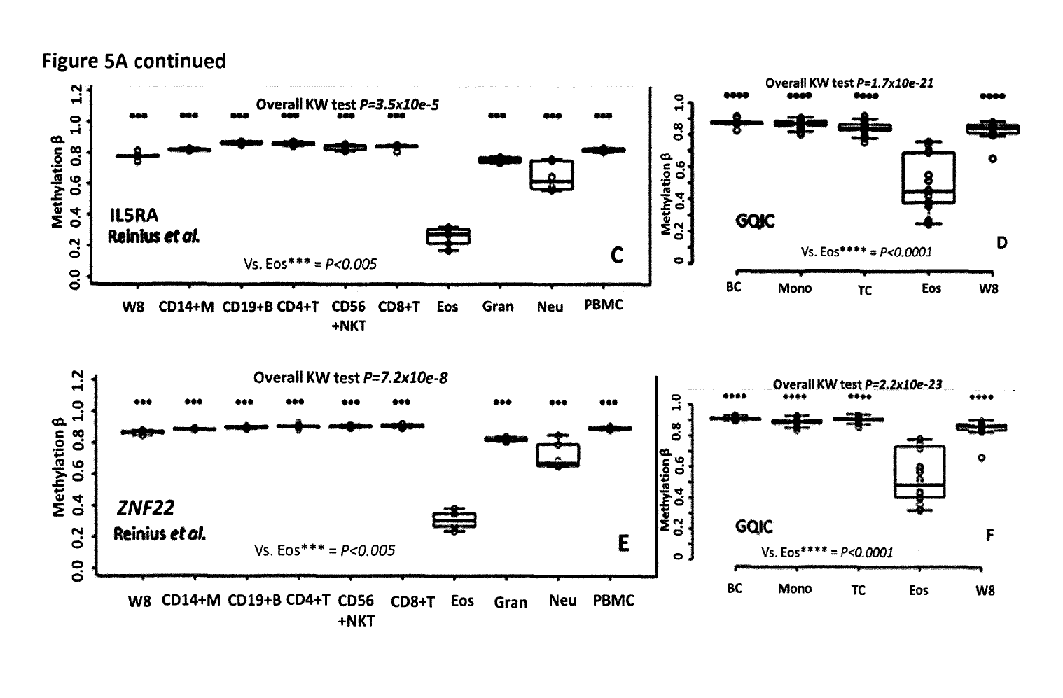

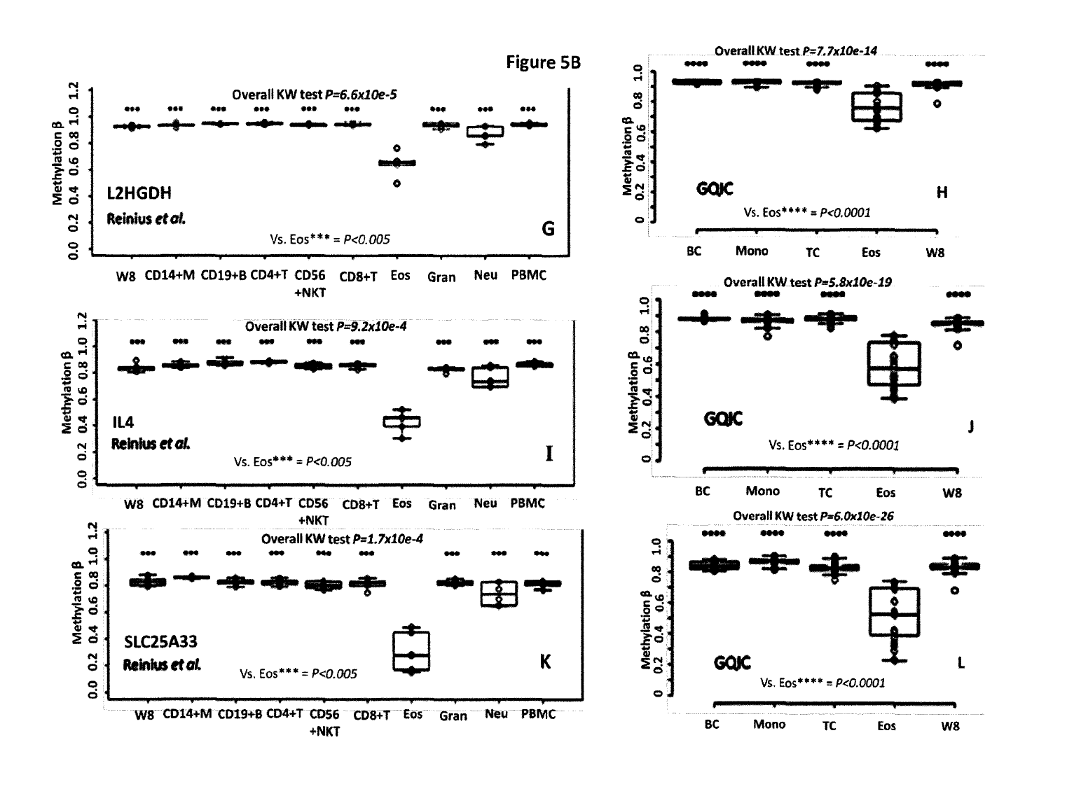

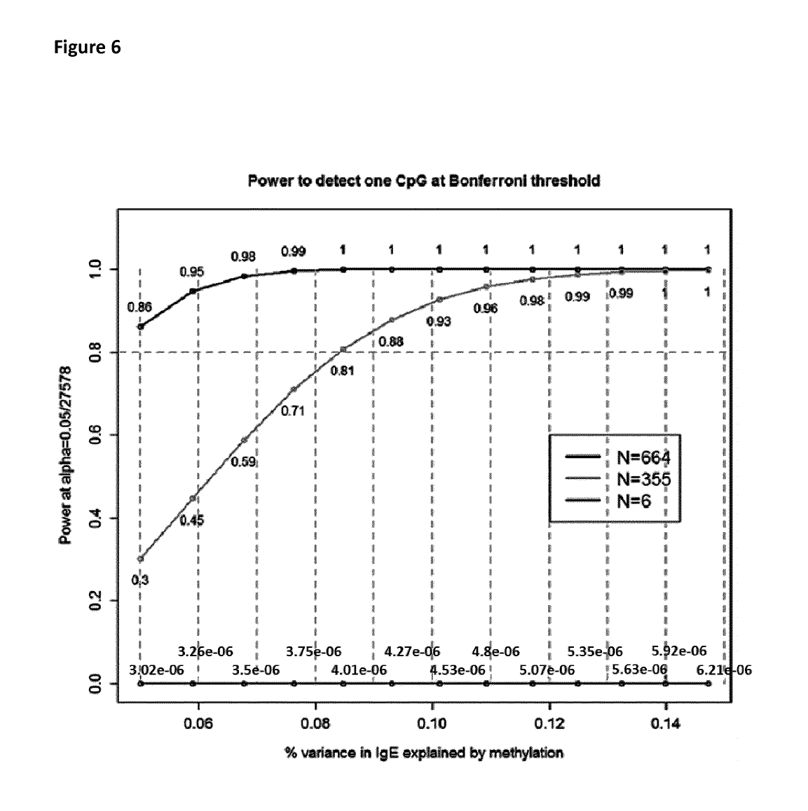

FIG. 4 shows a comparison between the present Illumina-based platform and whole genome bisulphite sequencing (WGBS). The results show a high R² between the two platforms (0.76 and 0.73). The median of the correlation coefficients for the IgE associated loci across 30 different samples (using WGBS at various depths) was R²=0.76. This result was similar to the global assessment of all overlapping 450K sites which was R²=0.81. Hence, these results establish that direct bisulphite pyrosequencing correlates robustly with the Illumina-based platform.

### Statistics

Age²⁵⁻²⁷, sex^{25,26}, genetic polymorphism^{28,29} and environmental factors^{27,29} have all been associated with altered methylation at selected loci. In order to investigate the association with the total serum IgE concentration, we tested for association with log-normalized IgE (Ln(IgE)) as response with each gene's methylation (β) as predictor whilst including batch indicators captured by Illumina chip ID and position of chip (such as operators, sample wells, plates, runs, and reagents), Sex, Age, Parent indicator, Age*Sex and Age*Parent interactions in the model. An inverse normal transformation on methylation measures was applied to remove the effect of outliers. The R function Ime( ) in the nIme package was used to implement a linear mixed model, assuming a compound symmetry variance-covariance structure to account for correlation of phenotypes among family members.

The R code for the discovery stage of association in the MRCA panel was:
index=!is.na(methylation)
fam=familyID[index]
par=parent[index]
methylation=methylation[index]
methylation=qnorm(rank(methylation)/(length(methylation)+1), mean=0, sd=1)
Inige=LNIGE[index]
age=AGE[index]
sex=SEX[index]
Im2=Ime(Inige^{˜}sex+age+methylation+par+sex*age+age*par, random=^{˜}1|fam)

The residual methylation values after removal of effects of chip ID and position for the genome-wide significant loci in the MRCA, PAPA and SLSJ panels, together with phenotypic and covariate parameters, are provided in Supplementary Tables 3 to 5 of “An Epigenome-Wide Association Study of Total Serum Immunoglobulin E Concentration”, Nature 2015 and Tables 9 to 11 of GB priority application no. 1423387.8, both incorporated herein by reference.

False discovery rates (FDR) were calculated and Bonferroni corrections were applied to adjust for multiple comparisons to 27,578 probes. The same analysis was carried out in the Swansea (PAPA) and SLSJ subjects before meta-analysis of the three studies. A weighted z-score method for meta-analysis was used, based on p value and effect direction from individual studies with weights proportion to the square root of sample size of each individual study⁵⁰. SNPs and indels using MINIMAC⁵¹. SNPs or indels with imputation quality score R²<0.3 were removed from downstream analysis. Mendelian randomization was used to assess the causal effect of IL-4 methylation on IgE level through a 2 stage last square instrumental variable regression⁵² implemented in the ivreg2.r program (http://diffusepriorwordpress.com/2012/05/03/an-ivreg2-function-for-r/).

Association trends were tested in isolated eosinophils by exact regression (Cytel Studio 9) with asthma/high IgE coded as 2, asthma/low IgE coded as 1, and controls as 0. Covariates for age, sex, and batch were included in the model and to test the hypothesis that low levels of methylation were associated with high IgE, P values were one-sided. Differences in methylation between peripheral blood leukocyte (PBL) subsets were assessed with Kruskal-Wallis tests, using two-sided P values.

### Results

The primary MRCA panel contained 355 subjects (183 male) with a mean age in children of 12.2 years (ranging from 2 to 39) and adults of 42 years (27 to 61). 113 of the children had doctor-diagnosed asthma (DDAST) (Table 1). The Swansea replication panel contained 149 subjects selected from the top and bottom deciles of the IgE distribution in 1614 unselected volunteers, and the SLSJ sample contained 160 subjects (80 male) with a mean age in children of 16 years (ranging from 5 to 50) and adults of 44 years (31 to 79). Forty of the children had doctor-diagnosed asthma (DDAST) (Table 1).

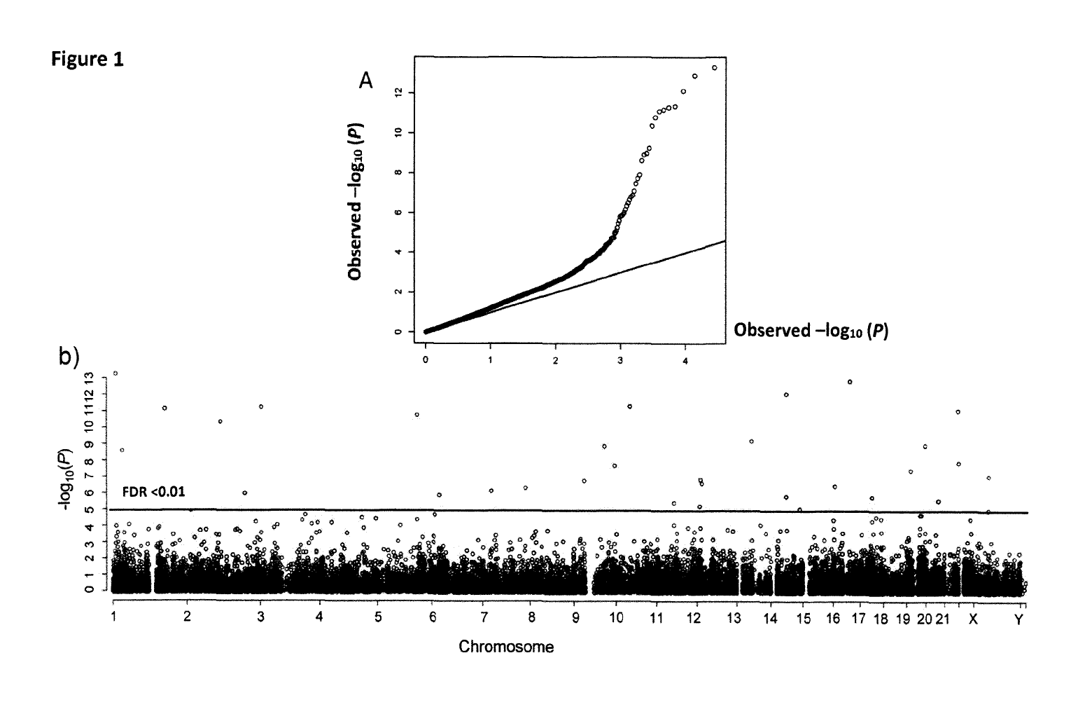

Models were initially fitted with Ln(IgE) as dependent variable and methylation status for each Illumina probe as a predictor with age, sex, parental status and interactions as covariates. 34 loci with FDR <0.01 (FIG. 1, Table 4 and Table 8) were identified in 32 different CGIs in the MRCA panel. Replication was sought in the Swansea and SLSJ panels, and a meta-analysis was then performed combining the results for all three panels. The meta-analysis identified 36 loci with FDR <10⁻⁴ and 69 probes with an FDR <0.01 (Table 2, Table 4 and Table 8). Replication was robust, with almost all loci from the MRCA panel showing significant associations with the same anti-correlated direction in the Swansea and SLSJ datasets (Table 2).

The most significant associations in the meta-analysis were LPCAT2 (Pₘₑₜₐ=1.2×10⁻¹⁸), IL5RA (Pₘₑₜₐ=2.2×10⁻¹⁸), ZNF22 (Pₘₑₜₐ=2.8×10⁻¹⁸), L2HGDH (Pₘₑₜₐ=2.8×10⁻¹⁷), IL4 (Pₘₑₜₐ=3.2×10⁻¹⁶), and SLC25A33 (Pₘₑₜₐ=4.4×10⁻¹⁵) (Table 2). Other significant loci were annotated to genes of known function in allergic inflammation, including the eosinophil granule major basic protein (PRG2) (Pₘₑₜₐ=3.1×10⁻⁹), the eosinophil transcription factor GATA1 (Pₘₑₜₐ=1.4×10⁻⁷), and the beta chain of the high affinity receptor for IgE (MS4A2) (Pₘₑₜₐ=2.0×10⁻⁶) (Table 4).

The possibility of methylation at these loci being related to allergen-specific IgE production was next examined. In the MRCA subjects, the RAST Index showed significant associations (FDR <0.01) with ZNF22 (P=6.5×10⁻⁸), LPCAT2 (P=1.4×10⁻⁷), L2HGDH (P=2.1×10⁻⁷), IL4 (P=2.2×10⁻⁷), IL5RA (P=3.5×10⁻⁷), SLC25A33 (P=8.7×10⁻⁷) and SDC3 (P=9.7×10⁻⁷). None of these remained significant if LnIgE was included as covariate in the model, suggesting that the loci had common effects on total and specific IgE production. No genome-wide significant associations to doctor-diagnosed asthma was found, but two of the top loci for IgE were also independently associated with asthma (LPCAT2, P=7.7×10⁻⁵ and ZNF22, P=1.8×10⁻⁴).

Lineage commitment to particular cell types can be defined by methylation status at specific loci,³⁰⁻³² and eosinophils and T_{H}2 lymphocytes may both contribute to IL4 production.^{6,7} To investigate if the associations to IgE reflected carriage in eosinophils or lymphocytes or other PBL cells, regression models in the MRCA subjects that included differential white cell counts were fitted. These models showed consistent independent associations with eosinophil numbers (Table 5), suggesting that eosinophils were primary carriers of the effects seen.

Methylation at CGIs associated with eosinophil counts (P<0.001) was then compared with published results of methylation status from isolated eosinophils from normal subjects.¹⁶ Strong correlations between the two datasets (R=0.64) were observed, suggesting an eosinophil derivation of the signals from these loci.

Surrogate CpG markers that identify lymphocyte subsets can be used as an alternative to white cell counts in association models⁵⁵. Hence these methods were also applied to our data (Table 7). This analysis provided further evidence that T-cell subsets do not have strong effects on these loci.

The variance (standard deviation) in our IgE-associated CpG loci was on average 4.4 fold larger in isolated eosinophils than in PBL from the MRCA dataset, indicating an attenuation of effect size in PBL that would mask associations rather than magnify them. The power to detect cell-specific associations from PBL depends on the proportion of each cell type, the effect size in specific cells, and the sample size. We estimate that we had 90% power to detect loci accounting for 10% variance in IgE in the MRCA panel and >99% power in the combined panels (FIG. 6).

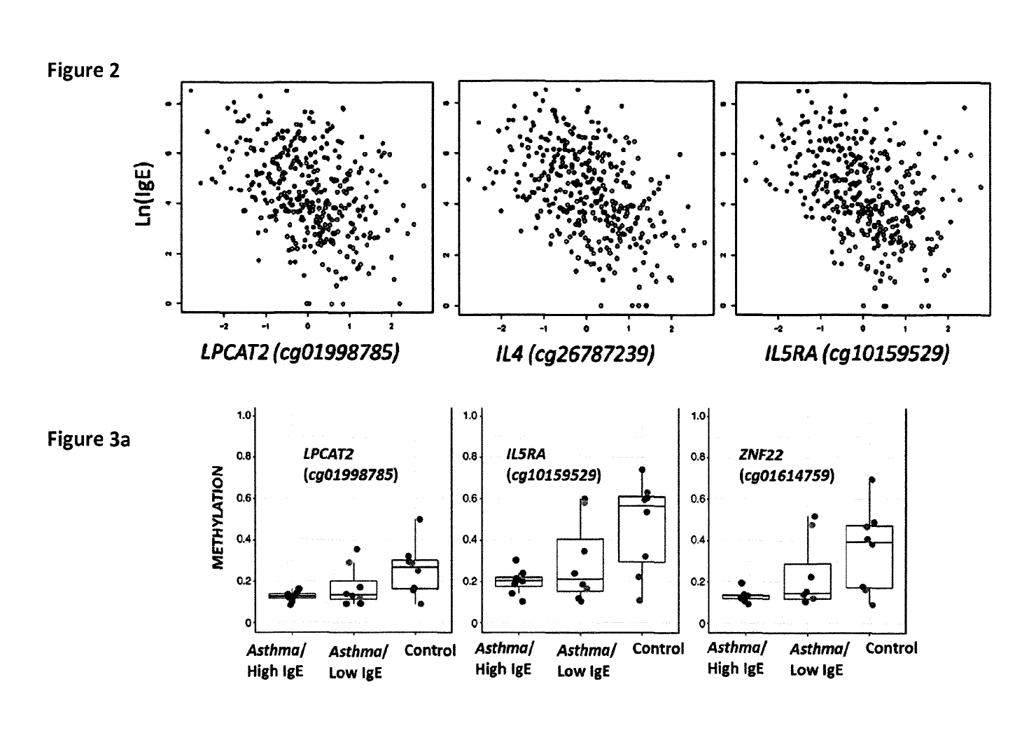

We partitioned plots of methylation status and LnIgE at the principal loci by eosinophil counts (FIG. 2), showing that the IgE associations are not confined to subjects with eosinophil counts above the median, and further confirming that methylation at these loci was not a simple surrogate for eosinophil numbers. We therefore hypothesised that CGI methylation at the loci captured events accompanying eosinophil activation.

In order to test this hypothesis, we purified eosinophils from peripheral blood from 8 asthmatics with high serum IgE levels (>110 IU/L), 8 asthmatics with low serum IgE (<110 IU/L) levels and 8 controls, all sub-selected from the SLSJ panel of subjects. The mean age of the subjects was 31 years (range 6-56), 8 subjects were female and 2 were current smokers. Asthmatics in both groups were on a maintenance regime of inhaled beta agonists, augmented with inhaled glucocorticoids during exacerbations.

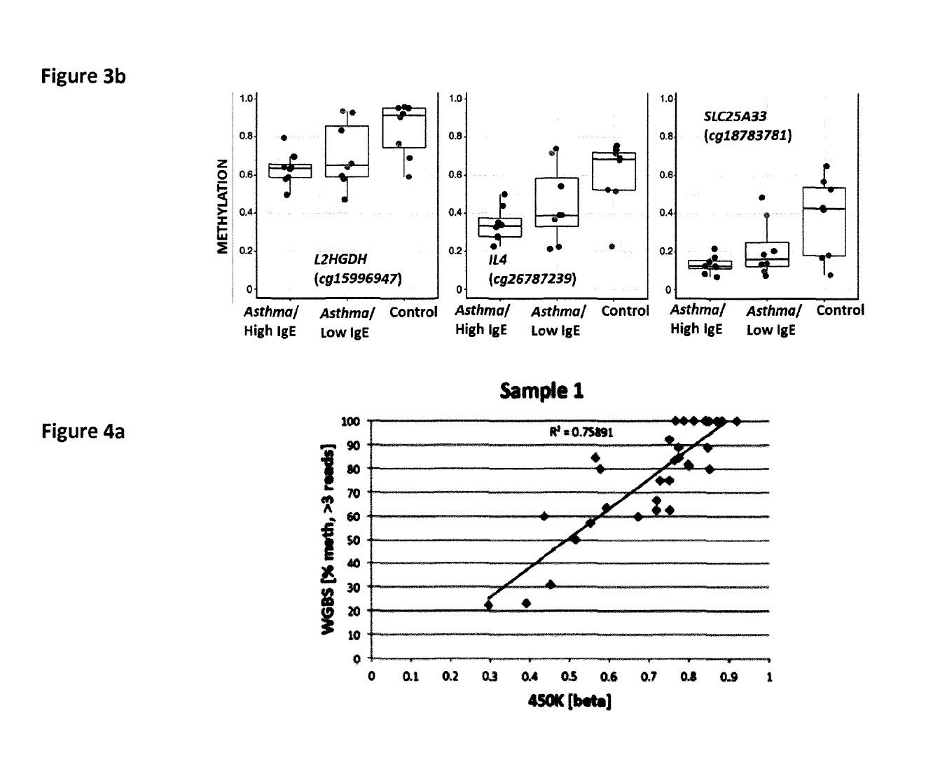

The range of variation for the principal loci appeared much narrower in asthmatics with high IgE (FIG. 3) than in the other two groups, consistent with the presence of a relatively homogeneous population of eosinophils in atopic asthma. We observed the lowest levels of methylation in the subjects with asthma and high IgE and that methylation in asthmatics with low IgE was intermediate to controls (FIG. 3) (trend test P<0.05; Table 4), in keeping with the results from the initial panels. Partitioning the data into high or low IgE groups gave similar conclusions.

The presence of positive associations at many loci suggested co-ordinated regulation of CGI methylation, which we investigated in the MRCA panel through a forward stepwise regression that included all significant CGI associations together with differential white cell counts, age, sex and parental status. This showed SLC25A33, LPCAT2 and L2HGDH to predict the total serum IgE concentration independently of each other and of eosinophil counts.

In the MRCA panel, the model estimated 13.5% of the variation in the total serum IgE to be attributable to these loci, whereas 8.8% of the variation was independently attributable to the eosinophil counts (Table 4). In the SLSJ panel, these three loci explained 8.3% IgE variation and 15.5% variation was attributable to eosinophil counts. The regression models therefore matched the results from isolated eosinophils, with the conclusion that the methylation status of eosinophils and their numbers were both related to IgE levels.

Although this analysis indicated the presence of independent CpG effects, similar estimations of variance were obtained with forced entry of other significantly associated markers into the models, so the results do not imply that SLC25A33, LPCAT2 and L2HGDH are the only biologically important loci.

In this respect, the variable methylation site upstream of IL4 which had a strong association to total serum IgE concentration has a well-studied major effect on IL4 production, T-cell lineage commitment to the T_{H}2 phenotype and subsequent IgE production^{37,38}, with decreasing methylation associated with increasing IgE in the same direction as that found in the present study. We looked for SNP associations at this locus by imputation with the 1000G phase 1 SNPs and indels in all three panels (i.e. MRCA, Swansea and SLSJ), analysing the 20746 variants within 1 Mb upstream or downstream of the IL4 5′UTR. There was no significant SNP association with IgE in the data obtained at the IL4 CGI¹⁸ after accounting for multiple testing, so the locus represents a functionally understood epigenetic association with a complex disease phenotype.

Mendelian randomization was next carried out to test for a causal effect of IL4 methylation on IgE⁴⁹, choosing the SNP showing strongest association to methylation at the IL4 CpG probe (cg26787239) as the instrumental variable. The First Stage F-test statistics for the MRCA and SLSJ panels (F=16.4 and 26.2) indicated effects strong enough to ensure the validity of the method. In the MRCA panel, association between the instrument SNP (rs12311504) and IgE9 before adjusting for IL4 methylation was P=0.03 and P=0.53 after adjustment, indicating that methylation mediated most of the SNP effect. The meta-analysis P for a causal effect was 6.8×10⁻⁴, suggesting that the locus represents a functionally validated epigenetic association with a complex phenotype.

In addition, IL5RA, CCR3 and IL1RL1 also have known signalling roles in allergic inflammation and also harboured altered methylation within their promoters that correlated with serum IgE levels. IL5 potently and specifically stimulates eosinophil production³⁹ and is a selective activator of human eosinophil function via binding to IL5RA⁴⁰. Notably, therapies directed against IL5 have already been shown to be effective in patients with eosinophilic asthma³⁶. CCR3 is the eosinophil eotaxin receptor, and IL1RL1 is the receptor for IL33.

Overall, the most significant association was to cg01998785, within a CGI adjacent to LPCAT2 (also known as AYTL1). LPCAT2 encodes lyso-PAF acetyltransferase, which is critical in stimulus-dependent formation of the potent pro-inflammatory lipid mediator PAF (Platelet-activating factor)⁴¹. It is of interest that hypoactive variants of plasmatic PAF-acetylhydrolase are associated with atopy and asthma⁴². Other significant associations were annotated to genes involved in phospholipid metabolism, including lysoplasmalogenase (TMEM86B), CEL and CLC.

The analyses also detected association to the known eosinophil transcription factor GATA1, but also suggest that the transcription factors ZNF22, RB1 and KLF may be important regulators of eosinophil activation. ZNF22 is of unknown function, RB1 mutations are commonly found in myeloid leukaemia, and KLF1 encodes an erythroid specific transcription factor.

Other significant associations may encode proteins released from eosinophil granules, including PRG2 (encoding eosinophil granule major basic protein), PRG3, SERPINC1 (antithrombin), TFF1 (which may protect the mucosa and stabilize the mucus layer), CEL (carboxyl ester lipase), and the polyvalent serine protease inhibitor SPINK4. Two of the most significant associations are to genes encoding mitochondrial proteins (L2HGDH and SLC25A33), perhaps reflecting mitochondrial regulation of apoptosis in activated eosinophils⁴³.

The above results are consistent with the recognition that eosinophils are an important source of cytokines and other pro-inflammatory molecules at the site of allergic inflammation⁷. Interestingly, it has been shown in mice that eosinophils are required locally for the maintenance of bone-marrow plasma cells,⁴⁴ providing a mechanism for their direct regulation of IgE production.

Taken together, the above results strongly suggest that the significant associations identified by the present inventors are likely to be clinically relevant targets for therapeutic intervention.

As the data obtained was array-based and quantitative batch and other potential occult experimental effects were explicitly modelled, and the results show that in the subjects tested, the variance in IgE attributable to CGI methylation was clearly independent of genetic variation. It thus seems likely to represent a response to environmental factors and it does not impact on the problem of missing heritability. Accordingly, this Example clearly demonstrates that the present inventors have identified robustly reproducible CGI associations that account for a substantial proportion of variation in total serum IgE that is 10-fold higher than that derived from large SNP genome wide association studies.

TABLE 1
Subjects and Populations

| | MRCA | Swansea | SLSJ |
|---|---|---|---|
| Number | 355 | 149 | 160 |
| Age (Mean, range) | 28, 2-61 | 21, 18-30 | 29, 5-79 |
| % Female | 172 (48.5%) | 72 (48.3%) | 80 (50.0%) |
| N (%) Asthmatic | 175 (49.3%) | 34 (22.8%) | 69 (43.1%) |
| N (%) Smoker | 45 (12.7%) | 33 (22.1%) | 28 (17.5%) |
| Eosinophil count | 0.41 +/− 0.38 | 0.25 +/− 0.21 | 0.24 +/− 0.21 |
| (mean +/− SE) | | | |
| Geometric Mean Serum | 320, 1-4999 | 663, 0-18800 | 412, 2-7653 |
| IgE (Range) IU | | | |

Numbers are shown for subjects who were successfully genotyped and whose genotypes passed all quality controls.

TABLE 2
Loci with genome-wide significance and tests of replication

| | | | P | | P | |
|---|---|---|---|---|---|---|
| Probe | Symbol | Function | MRCA | P SLSJ | Swans | P Meta |
| cg01998785 | LPCAT2 | Lysophospholipid metabolism | 1.2E−13 | 8.0E−03 | 9.6E−06 | 1.2E−18 |
| cg10159529 | IL5RA | Cytokine signalling | 5.1E−12 | 2.1E−04 | 7.2E−05 | 2.2E−18 |
| cg01614759 | ZNF22 | Transcription Factor | 4.4E−12 | 3.4E−03 | 2.8E−06 | 2.8E−18 |
| cg15996947 | L2HGDH | Mitochondrial oxidoreductase | 7.4E−13 | 1.0E−02 | 3.7E−05 | 2.8E−17 |
| cg26787239 | IL4 | Cytokine signalling | 1.6E−11 | 1.3E−03 | 4.8E−04 | 3.2E−16 |
| cg18783781 | SLC25A33 | Mitochondrial transport: dendritic cell | 5.0E−14 | 3.4E−02 | 6.2E−03 | 4.4E−15 |
| | | endocytosis | | | | |
| cg13221796 | RB1 | Transcription Factor | 5.7E−10 | 6.0E−02 | 4.9E−06 | 2.5E−14 |
| cg01770400 | SERPINC1 | Anti-thrombin | 6.6E−12 | 7.0E−03 | 1.3E−02 | 5.3E−14 |
| cg02643667 | TFF1 | Mucus stabilising secreted protein | 7.9E−12 | 1.3E−01 | 2.6E−03 | 7.6E−13 |
| cg21627181 | SLC17A4 | Sodium/phosphate cotransporter | 1.1E−06 | 1.4E−04 | 3.8E−04 | 1.1E−12 |
| cg20189937 | L2HGDH | Mitochondrial oxidoreductase | 1.3E−06 | 2.4E−03 | 3.2E−05 | 2.6E−12 |
| cg26457013 | TMEM86B | Lysoplasmalogenase: phospholipid metabolism | 1.0E−09 | 9.5E−02 | 9.0E−04 | 7.1E−12 |
| cg20503329 | COL15A1 | Cell shape motlity, adhesion | 1.2E−09 | 3.0E−01 | 8.8E−05 | 9.5E−12 |
| cg03693099 | CEL | Secreted carboxyl ester lipase | 1.8E−08 | 1.1E−01 | 8.3E−05 | 1.3E−11 |
| cg00079056 | SPINK4 | Serine peptidase inhibitor | 1.5E−07 | 6.4E−02 | 3.1E−05 | 1.8E−11 |
| cg09676390 | ADARB1 | Pre-mRNA editing of the glutamate receptor | 1.2E−08 | 7.0E−02 | 8.0E−04 | 3.1E−11 |
| cg15998761 | SEPT12 | Cell shape, motlity, adhesion | 9.3E−07 | 1.3E−02 | 1.7E−04 | 4.5E−11 |
| cg25494227 | TMEM52B | Transmembrane protein | 1.3E−07 | 2.0E−01 | 1.2E−05 | 5.1E−11 |
| cg11398517 | FAM112A | | 2.4E−06 | 7.4E−03 | 3.3E−04 | 1.0E−10 |
| cg06690548 | SLC7A11 | cystine/glutamate antiporter: dendritic cell | 2.7E−05 | 3.3E−04 | 1.1E−03 | 1.8E−10 |
| | | differentiation | | | | |
| cg17784922 | KEL | Metallo-endopeptidase | 4.2E−07 | 7.9E−03 | 4.4E−03 | 2.1E−10 |
| cg16050349 | PIK3CB | Catalytic subunit for PI3Kbeta: activation of | 4.0E−05 | 1.7E−03 | 2.3E−04 | 3.2E−10 |
| | | neutrophils | | | | |
| cg25636075 | TMEM41A | Transmembrane protein | 2.5E−04 | 5.1E−05 | 7.7E−04 | 3.9E−10 |
| cg08404225 | IL5RA | Cytokine signalling | 2.3E−04 | 3.3E−03 | 8.4E−06 | 4.1E−10 |
| cg09447105 | PDE6H | Inhibitory subunit of cGMP phosphodiesterase | 2.2E−07 | 1.3E−01 | 4.0E−04 | 5.3E−10 |
| cg05215575 | SEPT12 | Cell shape, motlity, adhesion | 3.1E−07 | 2.1E−01 | 2.7E−04 | 1.2E−09 |
| cg26136776 | KLF1 | Erythroid-specific transcription factor | 3.3E−08 | 4.3E−01 | 6.6E−04 | 1.5E−09 |
| cg17749520 | ITGA2B | Platelet fibronectin receptor: role in coagulation | 1.4E−06 | 2.5E−02 | 3.5E−03 | 1.8E−09 |
| cg24459209 | PRG3 | eosinophil major basic protein homolog | 3.3E−06 | 2.9E−02 | 1.1E−03 | 1.8E−09 |
| cg00002426 | SLMAP | Sarcolemma associated protein | 7.9E−05 | 8.3E−03 | 1.6E−04 | 2.4E−09 |
| cg15357945 | PRG2 | Eosinophll granule major basic protein | 2.2E−03 | 2.8E−05 | 5.8E−04 | 3.1E−09 |
| cg17582777 | EFNA3 | Receptor protein-tyrosine kinase | 1.1E−04 | 3.1E−02 | 8.2E−05 | 8.6E−09 |
| cg19881895 | SLC43A3 | Transmembrane protein | 7.5E−05 | 2.8E−03 | 6.7E−03 | 1.6E−08 |
| cg18254848 | CLC | Lysophospholipid metabolism | 1.8E−05 | 4.4E−02 | 4.6E−03 | 4.5E−08 |
| cg21631409 | ALDH3B2 | Enzyme or Kinase | 2.3E−04 | 1.7E−02 | 1.2E−03 | 6.8E−08 |
| cg00536175 | GATA1 | Eosinophil transcription factor | 7.9E−08 | 4.0E−01 | 5.1E−02 | 1.4E−07 |
| cg04111761 | CCR3 | eotaxin receptor | 1.2E−04 | 2.3E−01 | 1.6E−04 | 2.1E−07 |
| cg16386158 | IL1RL1 | IL33 receptor | 1.0E−03 | 6.6E−02 | 1.5E−04 | 3.3E−07 |

Loci with a false discovery rate for the meta-analysis <10⁻⁴. Full list of significant associations are shown in Table 4. Markers are identified through their Illumina IDs and the associated Gene symbol is derived from the Illumina annotation updated through PubMed. Note that two probes from IL5RA and from L2HGDH are associated to IgE concentrations.

TABLE 3
Details of Probes tested

| | | Genome | | | | Source | TSS | Gene | | | Distance | | |
|---|---|---|---|---|---|---|---|---|---|---|---|---|---|
| Probe | Symbol | Build | Chr | MapInfo | Source | Version | Coordinate | Strand | Gene_ID | Symbol | to TSS | CPG_ISLAND | CPG_ISLAND_LOCATIONS |
| cg01998785 | LPCAT2 | 36 | 16 | 54100210 | NCBI: RefSeq | 36.1 | 54100455 | + | GeneID: 54947 | AYTL1 | 245 | FALSE | — |
| cg10159529 | IL5RA | 36 | 3 | 3127530 | NCBI: RefSeq | 36.1 | 3127031 | − | GeneID: 3568 | IL5RA | 499 | FALSE | — |
| cg01614759 | ZNF22 | 36 | 10 | 44815441 | NCBI: RefSeq | 36.1 | 44815928 | + | GeneID: 7570 | ZNF22 | 487 | FALSE | — |
| cg15996947 | L2HGDH | 36 | 14 | 49849865 | NCBI: RefSeq | 36.1 | 49848697 | − | GeneID: 79944 | L2HGDH | 1168 | FALSE | — |
| cg26787239 | IL4 | 36 | 5 | 1.32E+08 | NCBI: RefSeq | 36.1 | 1.32E+08 | + | GeneID: 3565 | IL4 | 848 | FALSE | — |
| cg18783781 | SLC25A33 | 36 | 1 | 9521654 | NCBI: RefSeq | 36.1 | 9522145 | + | GeneID: 84275 | MGC4399 | 491 | TRUE | 1:9521329-9523202 |
| cg13221796 | RB1 | 36 | 13 | 47774920 | NCBI: RefSeq | 36.1 | 47775912 | + | GeneID: 5925 | RB1 | 992 | FALSE | — |
| cg01770400 | SERPINC1 | 36 | 1 | 1.72E+08 | NCBI: RefSeq | 36.1 | 1.72E+08 | − | GeneID: 462 | SERPINC1 | 12 | FALSE | — |
| cg02643667 | TFF1 | 36 | 21 | 42659768 | NCBI: RefSeq | 36.1 | 42659713 | − | GeneID: 7031 | TFF1 | 55 | FALSE | — |
| cg21627181 | SLC17A4 | 36 | 6 | 25862169 | NCBI: RefSeq | 36.1 | 25862945 | + | GeneID: 10050 | SLC17A4 | 776 | FALSE | — |
| cg20189937 | L2HGDH | 36 | 14 | 49849874 | NCBI: RefSeq | 36.1 | 49848697 | − | GeneID: 79944 | L2HGDH | 1177 | FALSE | — |
| cg26457013 | TMEM86B | 36 | 19 | 60432000 | NCBI: RefSeq | 36.1 | 60432444 | − | GeneID: 255043 | TMEM86B | 444 | FALSE | — |
| cg20503329 | COL15A1 | 36 | 9 | 1.01E+08 | NCBI: RefSeq | 36.1 | 1.01E+08 | + | GeneID: 1306 | COL15A1 | 398 | TRUE | 9:100745603-100747003 |
| cg03693099 | CEL | 36 | 9 | 1.35E+08 | NCBI: RefSeq | 36.1 | 1.35E+08 | + | GeneID: 1056 | CEL | 464 | FALSE | — |
| cg00079056 | SPINK4 | 36 | 9 | 33229641 | NCBI: RefSeq | 36.1 | 33230196 | + | GeneID: 27290 | SPINK4 | 555 | FALSE | — |
| cg09676390 | ADARB1 | 36 | 21 | 45317773 | NCBI: RefSeq | 36.1 | 45318943 | + | GeneID: 104 | ADARB1 | 1170 | TRUE | 21:45317770-45320123 |
| cg15998761 | FLJ20160 | 36 | 2 | 1.91E+08 | NCBI: RefSeq | 36.1 | 1.91E+08 | + | GeneID: 54842 | FLJ20160 | 85 | FALSE | — |
| cg25494227 | TMEM52B | 36 | 12 | 10222881 | NCBI: RefSeq | 36.1 | 10222898 | + | GeneID: 120939 | C12orf59 | 17 | FALSE | — |
| cg11398517 | FAM112A | 36 | 20 | 41789039 | NCBI: RefSeq | 36.1 | 41789056 | − | GeneID: 149699 | FAM112A | 17 | FALSE | — |
| cg06690548 | SLC7A11 | 36 | 4 | 1.39E+08 | NCBI: RefSeq | 36.1 | 1.39E+08 | − | GeneID: 23657 | SLC7A11 | 415 | TRUE | 4:139382255-139382463 |
| cg17784922 | KEL | 36 | 7 | 1.42E+08 | NCBI: RefSeq | 36.1 | 1.42E+08 | − | GeneID: 3792 | KEL | 78 | FALSE | — |
| cg16050349 | PIK3CB | 36 | 3 | 1.4E+08 | NCBI: RefSeq | 36.1 | 1.4E+08 | − | GeneID: 5291 | PIK3CB | 56 | FALSE | — |
| cg25636075 | TMEM41A | 36 | 3 | 1.87E+08 | NCBI: RefSeq | 36.1 | 1.87E+08 | − | GeneID: 90407 | TMEM41A | 959 | TRUE | 3:186700380-186700792 |
| cg08404225 | IL5RA | 36 | 3 | 3126899 | NCBI: RefSeq | 36.1 | 3127031 | − | GeneID: 3568 | IL5RA | 132 | FALSE | — |
| cg09447105 | PDE6H | 36 | 12 | 15017287 | NCBI: RefSeq | 36.1 | 15017245 | + | GeneID: 5149 | PDE6H | 42 | FALSE | — |
| cg05215575 | SEPT12 | 36 | 16 | 4778723 | NCBI: RefSeq | 36.1 | 4778348 | − | GeneID: 124404 | FL125410 | 375 | FALSE | — |
| cg26136776 | KLF1 | 36 | 19 | 12859426 | NCBI: RefSeq | 36.1 | 12859017 | − | GeneID: 10661 | KFL1 | 409 | FALSE | — |
| cg17749520 | ITGA2B | 36 | 17 | 39822093 | NCBI: RefSeq | 36.1 | 39822399 | − | GeneID: 3674 | ITGA2B | 306 | FALSE | — |
| cg24459209 | PRG3 | 36 | 11 | 56904791 | NCBI: RefSeq | 36.1 | 56905199 | − | GeneID: 10394 | PRG3 | 408 | FALSE | — |
| cg00002426 | SLMAP | 36 | 3 | 57718583 | NCBI: RefSeq | 36.1 | 57718214 | + | GeneID: 7871 | SLMAP | 369 | TRUE | 3:57716811-57718675 |
| cg15357945 | PRG2 | 36 | 11 | 56914937 | NCBI: RefSeq | 36.1 | 56914706 | − | GeneID: 5553 | PRG2 | 231 | FALSE | — |
| cg17582777 | EFNA3 | 36 | 1 | 1.53E+08 | NCBI: RefSeq | 36.1 | 1.53E+08 | + | GeneID: 1944 | EFNA3 | 1248 | FALSE | — |
| cg19881895 | SLC43A3 | 36 | 11 | 56952116 | NCBI: RefSeq | 36.1 | 56951629 | − | GeneID: 29015 | SLC43A3 | 487 | FALSE | — |
| cg18254848 | CLC | 36 | 19 | 44919789 | NCBI: RefSeq | 36.1 | 44920508 | − | GeneID: 1178 | CLC | 719 | FALSE | — |
| cg21631409 | ALDH3B2 | 36 | 11 | 67206458 | NCBI: RefSeq | 36.1 | 67205261 | − | GeneID: 222 | ALDH3B2 | 1197 | FALSE | — |
| cg00536175 | GATA1 | 36 | X | 48529968 | NCBI: RefSeq | 36.1 | 48529906 | + | GeneID: 2623 | GATA1 | 62 | FALSE | — |
| cg04111761 | CCR3 | 36 | 3 | 46257770 | NCBI: RefSeq | 36.1 | 46258692 | + | GeneID: 1232 | CCR3 | 922 | FALSE | — |
| cg16386158 | IL1RL1 | 36 | 2 | 1.02E+08 | NCBI: RefSeq | 36.1 | 1.02E+08 | + | GeneID: 9173 | IL1RL1 | 555 | FALSE | — |

| | |
|---|---|
| Name | CG number from CG database (format cg########) |
| GenomeBuild | Genome build |
| Chr | Chromosome on which the target locus is located |
| Mapinfo | Genomic position of C in CG dinucleotide |
| Source | Genomic position source |
| SourceVersion | Source version |
| TSS_Coordinate | Transcription start site genomic coordinate |
| Gene_Strand | Gene strand |
| Gene_ID | RefSeq identifier (GeneID) |
| Symbol | Gene Symbol |
| Distance_to_TSS | Distance of CG dinucleotide to transcription start site |
| CPG_ISLAND | Boolean variable denoting whether the loci is located in a CpG island (by relaxed definition) |
| CPG_ISLAND_LOCATIONS | Chromosomal location and genomic coordonates of the CpG island from NCBI database |
| MIR_CPG_ISLAND | Chromosome: start-end of upstream CPG island from a micro RNA |
| MIR_NAMES | Name of micro RNA near locus |

TABLE 4
LnIgE associations with CGI in three populations with meta-analysis; and in isolated eosinophils

| Probe | Symbol | P MRCA | e MRCA | p McGill | e McGill | P Swansea | e Swansea | p Meta | P Isolated |
|---|---|---|---|---|---|---|---|---|---|
| cg01998785 | LPCAT2 | 1.24E−13 | −0.59569 | 0.008026 | −0.40092 | 9.60E−06 | −1.4627 | 1.16E−18 | 0.042 |
| cg10159529 | IL5RA | 5.06E−12 | −0.56178 | 0.000208 | −0.5518 | 7.25E−05 | −1.33591 | 2.17E−18 | 0.030 |
| cg01614759 | ZNF22 | 4.42E−12 | −0.55724 | 0.003444 | −0.41241 | 2.81E−06 | −1.79231 | 2.83E−18 | 0.037 |
| cg15996947 | L2HGDH | 7.39E−13 | −0.60592 | 0.010325 | −0.3655 | 3.73E−05 | −1.20484 | 2.75E−17 | 0.034 |
| cg26787239 | IL4 | 1.59E−11 | −0.56341 | 0.001265 | −0.43654 | 0.000479 | −1.06348 | 3.24E−16 | 0.026 |
| cg18783781 | SLC25A33 | 4.96E−14 | −0.62716 | 0.03433 | −0.30222 | 0.006186 | −0.68378 | 4.43E−15 | 0.025 |
| cg13221796 | RB1 | 5.66E−10 | −0.50355 | 0.059619 | −0.2518 | 4.95E−06 | −1.6967 | 2.49E−14 | 0.076 |
| cg01770400 | SERPINC1 | 6.59E−12 | −0.55738 | 0.006959 | −0.37242 | 0.012631 | −0.63027 | 5.27E−14 | 0.026 |
| cg02643667 | TFF1 | 7.87E−12 | −0.59305 | 0.131609 | −0.21306 | 0.002596 | −0.98624 | 7.59E−13 | 0.003 |
| cg21627181 | SLC17A4 | 1.14E−06 | −0.39818 | 0.000138 | −0.571 | 0.000378 | −1.03867 | 1.13E−12 | 0.034 |
| cg20189937 | L2HGDH | 1.32E−06 | −0.38816 | 0.002367 | −0.41629 | 3.19E−05 | −1.68808 | 2.55E−12 | 0.033 |
| cg26457013 | TMEM86B | 1.02E−09 | −0.49167 | 0.095316 | −0.23286 | 0.0009 | −0.89216 | 7.06E−12 | 0.021 |
| cg20503329 | COL15A1 | 1.19E−09 | −0.48099 | 0.300135 | −0.13391 | 8.83E−05 | −1.24104 | 9.50E−12 | 0.015 |
| cg03693099 | CEL | 1.84E−08 | −0.47488 | 0.108643 | −0.21581 | 8.30E−05 | −1.35572 | 1.32E−11 | 0.023 |
| cg00079056 | SPINK4 | 1.54E−07 | −0.42211 | 0.063911 | −0.26672 | 3.13E−05 | −1.36429 | 1.81E−11 | 0.013 |
| cg09676390 | ADARB1 | 1.21E−08 | −0.4931 | 0.070175 | −0.2652 | 0.000803 | −0.90873 | 3.06E−11 | 0.024 |
| cg15998761 | FLJ20160 | 9.33E−07 | −0.39912 | 0.013172 | −0.34934 | 0.000171 | −1.18429 | 4.55E−11 | 0.030 |
| cg25494227 | C12orf59 | 1.29E−07 | −0.44273 | 0.199089 | −0.17655 | 1.18E−05 | −1.47126 | 5.09E−11 | 0.024 |
| cg11398517 | FAM112A | 2.42E−06 | −0.38183 | 0.007404 | −0.43205 | 0.000328 | −1.06414 | 1.02E−10 | 0.014 |
| cg06690548 | SLC7A11 | 2.75E−05 | −0.3424 | 0.00033 | −0.51241 | 0.00107 | −0.87874 | 1.79E−10 | 0.042 |
| cg17784922 | KEL | 4.24E−07 | −0.39655 | 0.007882 | −0.36644 | 0.004403 | −0.72455 | 2.14E−10 | 0.013 |
| cg16050349 | PIK3CB | 4.03E−05 | −0.33564 | 0.001743 | −0.43169 | 0.000225 | −1.01162 | 3.23E−10 | 0.025 |
| cg25636075 | TMEM41A | 0.000252 | −0.29715 | 5.06E−05 | −0.5839 | 0.000772 | −1.01626 | 3.86E−10 | 0.019 |
| cg08404225 | IL5RA | 0.00023 | −0.3048 | 0.003271 | −0.4285 | 8.41E−06 | −1.70975 | 4.11E−10 | 0.028 |
| cg09447105 | PDE6H | 2.16E−07 | −0.41885 | 0.130267 | −0.23289 | 0.000404 | −1.04935 | 5.27E−10 | 0.020 |
| cg05215575 | FLJ25410 | 3.11E−07 | −0.43076 | 0.210279 | −0.18076 | 0.000272 | −1.16021 | 1.19E−09 | 0.306 |
| cg26136776 | KLF1 | 3.34E−08 | −0.44803 | 0.429565 | −0.10678 | 0.000661 | −0.95909 | 1.54E−09 | 0.040 |
| cg17749520 | ITGA2B | 1.37E−06 | −0.42943 | 0.024723 | −0.33917 | 0.003466 | −0.84297 | 1.76E−09 | 0.008 |
| cg24459209 | PRG3 | 3.33E−06 | −0.38834 | 0.02945 | −0.315 | 0.001105 | −0.85001 | 1.81E−09 | 0.022 |
| cg00002426 | SLMAP | 7.92E−05 | −0.32642 | 0.008326 | −0.38564 | 0.000162 | −1.20425 | 2.38E−09 | 0.017 |
| cg15357945 | PRG2 | 0.002214 | −0.28738 | 2.82E−05 | −0.60297 | 0.000581 | −0.94557 | 3.14E−09 | 0.044 |
| cg17582777 | EFNA3 | 0.000107 | −0.32224 | 0.031154 | −0.29045 | 8.22E−05 | −1.2615 | 8.56E−09 | 0.017 |
| cg19881895 | SLC43A3 | 7.50E−05 | −0.31886 | 0.002787 | −0.40199 | 0.006735 | −0.6856 | 1.63E−08 | 0.028 |
| cg18254848 | CLC | 1.77E−05 | −0.3492 | 0.04441 | −0.28497 | 0.004566 | −0.72532 | 4.52E−08 | 0.016 |
| cg21631409 | ALDH3B2 | 0.000234 | −0.29132 | 0.016894 | −0.32961 | 0.001223 | −0.86863 | 6.83E−08 | 0.036 |
| cg00536175 | GATA1 | 7.85E−08 | −0.43636 | 0.401655 | −0.14262 | 0.050597 | −0.55841 | 1.41E−07 | 0.026 |
| cg04111761 | CCR3 | 0.000117 | −0.3337 | 0.233512 | −0.17448 | 0.000162 | −1.26197 | 2.10E−07 | 0.026 |
| cg16386158 | IL1RL1 | 0.001016 | −0.26235 | 0.065953 | −0.27399 | 0.000148 | −1.08979 | 3.31E−07 | 0.029 |
| cg12866859 | HEXIM1 | 4.09E−05 | −0.33394 | 0.029764 | −0.31863 | 0.030243 | −0.60308 | 3.54E−07 | |
| cg26251865 | IRGC | 1.76E−05 | −0.35144 | 0.221901 | −0.16501 | 0.004974 | −0.79576 | 3.97E−07 | |
| cg17890764 | ITIH4 | 0.001863 | −0.27823 | 0.06846 | −0.27451 | 9.81E−05 | −1.07967 | 5.25E−07 | |
| cg16522484 | C14orf49 | 8.04E−06 | −0.3588 | 0.449419 | −0.11511 | 0.007388 | −0.72584 | 9.34E−07 | |
| cg08377000 | TIGD2 | 0.000244 | −0.30374 | 0.080931 | −0.23267 | 0.004338 | −0.76764 | 1.00E−06 | |
| cg13424229 | CPA3 | 1.89E−05 | −0.38251 | 0.083486 | −0.23337 | 0.068455 | −0.52171 | 1.30E−06 | |
| cg26385286 | GCNT2 | 1.78E−05 | −0.34608 | 0.877289 | −0.02145 | 0.000646 | −0.92038 | 1.36E−06 | |
| cg10805676 | MRPL28 | 3.73E−05 | −0.35955 | 0.305084 | −0.1427 | 0.008461 | −0.60543 | 1.87E−06 | |
| cg27653134 | A2ML1 | 0.00243 | −0.23973 | 0.229057 | −0.17252 | 4.21E−05 | −1.3361 | 2.03E−06 | |
| cg10414946 | MS4A2 | 0.010495 | −0.23379 | 0.005758 | −0.43389 | 0.001316 | −0.9326 | 2.04E−06 | |
| cg10280342 | PSPN | 0.000397 | −0.28369 | 0.026299 | −0.32857 | 0.041761 | −0.55638 | 3.41E−06 | |
| cg16396488 | PLA2G1B | 0.000178 | −0.30652 | 0.392381 | −0.12128 | 0.001876 | −0.84923 | 3.58E−06 | |
| cg23759710 | OXER1 | 0.000179 | −0.30704 | 0.25365 | −0.1612 | 0.006281 | −0.74809 | 4.31E−06 | |
| cg07689731 | SDC3 | 2.33E−09 | −0.48819 | 0.284739 | −0.14857 | 0.49682 | 0.181103 | 4.97E−06 | |
| cg09793866 | STAR | 0.047481 | −0.16692 | 0.013517 | −0.32562 | 7.28E−05 | −1.17221 | 5.53E−06 | |
| cg06736444 | SRRM2 | 0.000544 | −0.29122 | 0.062683 | −0.25598 | 0.024796 | −0.60662 | 6.62E−06 | |
| cg20967028 | ART4 | 0.000578 | −0.28835 | 0.170734 | −0.18059 | 0.007478 | −0.67519 | 8.31E−06 | |
| cg21682902 | HAL | 0.008723 | −0.22446 | 0.037814 | −0.29881 | 0.001378 | −0.93095 | 8.41E−06 | |
| cg04523589 | CAMP | 0.001075 | −0.28064 | 0.114586 | −0.21991 | 0.006842 | −0.71468 | 8.68E−06 | |
| cg03014680 | CLEC12A | 0.000111 | −0.31709 | 0.174865 | −0.18646 | 0.046444 | −0.50304 | 9.17E−06 | |
| cg23064554 | CTRC | 0.001929 | −0.25292 | 0.049392 | −0.2489 | 0.011653 | −0.65792 | 9.52E−06 | |
| cg00596686 | STS | 0.013118 | −0.22173 | 0.049665 | −0.35212 | 0.000548 | −1.10548 | 1.00E−05 | |
| cg07374928 | FLJ21103 | 0.000199 | 0.288243 | 0.04533 | 0.269185 | 0.149386 | 0.343544 | 1,16E−05 | |
| cg03580247 | SLC4A1 | 1.51E−06 | −0.4025 | 0.579819 | −0.07376 | 0.214892 | −0.32745 | 1.21E−05 | |
| cg06394229 | LGALS4 | 0.039225 | −0.17088 | 0.006981 | −0.36507 | 0.00152 | −0.84874 | 1.45E−05 | |
| cg22543648 | GATA1 | 9.51E−06 | −0.46481 | 0.816783 | −0.03134 | 0.043789 | −0.53224 | 1.66E−05 | |
| cg05154390 | MRPS15 | 0.000247 | −0.36152 | 0.133717 | −0.26479 | 0.065775 | −0.47829 | 1.81E−05 | |
| cg12818699 | C6orf32 | 0.002133 | −0.28352 | 0.23043 | −0.18967 | 0.002332 | −0.78112 | 1.89E−05 | |
| cg05869585 | PMM2 | 0.007574 | −0.21217 | 0.335599 | −0.13831 | 0.00013 | −1.15359 | 2.23E−05 | |
| cg11136251 | ZWILCH | 0.002756 | 0.249365 | 0.045538 | 0.255219 | 0.024543 | 0.63765 | 2.27E−05 | |
| cg09914444 | DMBX1 | 0.010512 | −0.23967 | 0.003648 | −0.4455 | 0.050059 | −0.54119 | 2.38E−05 | |
| cg05637892 | SCFD1 | 0.000163 | 0.332484 | 0.500688 | 0.091338 | 0.021117 | 0.570384 | 2.91E−05 | |
| cg04881903 | CAPG | 0.022839 | −0.19279 | 0.075439 | −0.24832 | 0.000534 | −0.90867 | 2.92E−05 | |
| cg12894629 | OSTalpha | 0.000218 | −0.29503 | 0.291738 | −0.15645 | 0.048649 | −0.50561 | 3.25E−05 | |
| cg24670715 | ANGPT2 | 0.000483 | −0.29297 | 0.227293 | −0.1682 | 0.035548 | −0.52605 | 3.47E−05 | |
| cg15827295 | LYSMD1 | 0.002092 | 0.237693 | 0.054969 | 0.25354 | 0.045847 | 0.532073 | 3.51E−05 | |
| cg27429194 | OR1A2 | 0.002958 | 0.238678 | 0.11398 | 0.285094 | 0.01347 | 0.82647 | 3.78E−05 | |
| cg26718420 | C12orf59 | 0.002567 | −0.24065 | 0.297502 | −0.13595 | 0.004209 | −0.7672 | 4.63E−05 | |
| cg24988345 | SCHIP1 | 0.0201 | −0.18819 | 0.010394 | −0.33337 | 0.021761 | −0.60149 | 5.23E−05 | |
| cg00298951 | CMKLR1 | 0.017468 | −0.19615 | 0.042985 | −0.29247 | 0.006158 | −0.8657 | 5.57E−05 | |
| cg07173760 | CLC | 0.000107 | −0.32493 | 0.713991 | 0.052885 | 0.003828 | −0.7791 | 5.70E−05 | |
| cg14849423 | PEG3 | 0.000223 | 0.29329 | 0.54886 | 0.082049 | 0.029634 | 0.598817 | 5.72E−05 | |
| cg11584111 | PIGC | 0.010959 | 0.233367 | 0.035813 | 0.263402 | 0.020612 | 0.553657 | 6.67E−05 | |
| cg24631950 | UBE2D1 | 0.000757 | −0.26009 | 0.06372 | −0.24863 | 0.206282 | −0.31482 | 7.17E−05 | |
| cg23504707 | PPM1A | 0.147206 | −0.11624 | 3.71E−05 | −0.53934 | 0.062622 | −0.45537 | 7.29E−05 | |
| cg20622019 | ADA | 0.081326 | 0.140387 | 0.052998 | 0.260096 | 0.000335 | 1.043897 | 8.63E−05 | |
| cg27316956 | SYNE1 | 0.021032 | −0.19551 | 0.001345 | −0.50104 | 0.162657 | −0.3674 | 8.79E−05 | |
| cg27214365 | GYPB | 0.001287 | −0.25797 | 0.322595 | −0.14222 | 0.024032 | −0.59639 | 9.28E−05 | |
| cg10635061 | FHL2 | 0.000319 | −0.28789 | 0.421648 | −0.11568 | 0.062819 | −0.46918 | 9.32E−05 | |
| cg18338293 | BBS1 | 0.001825 | −0.25243 | 0.647452 | −0.0638 | 0.003197 | −0.80618 | 9.53E−05 | |
| cg01656750 | KATNB1 | 0.011586 | −0.21783 | 0.06109 | −0.27325 | 0.016669 | −0.5974 | 9.61E−05 | |
| c827016609 | STON1 | 0.24574 | −0.09313 | 0.000116 | −0.60671 | 0.016576 | −0.60093 | 0.000106 | |
| cg05064181 | ABLIM1 | 0.002758 | −0.24135 | 0.126971 | −0.19235 | 0.052314 | −0.50415 | 0.000115 | |
| cg07237830 | BSCL2 | 0.020099 | −0.18921 | 0.083958 | −0.24495 | 0.006013 | −0.71316 | 0.000118 | |
| cg04848046 | FNDC3B | 0.026137 | −0.18648 | 0.024172 | −0.30489 | 0.018818 | −0.58121 | 0.00012 | |
| cg27094188 | EIF2C1 | 0.003372 | −0.23371 | 0.07617 | −0.23943 | 0.080273 | −0.42056 | 0.000122 | |
| cg04180953 | DSC1 | 0.006641 | −0.21837 | 0.145588 | −0.1943 | 0.015976 | −0.6265 | 0.000122 | |
| cg03221619 | FCER2 | 0.000522 | 0.290929 | 0.222375 | 0.174773 | 0.153 | 0.367372 | 0.000138 | |
| cg05155595 | ANXA4 | 0.011323 | −0.2557 | 0.324108 | −0.1463 | 0.001935 | −0.87691 | 0.000141 | |
| cg07336230 | KIF6 | 0.007529 | −0.23132 | 0.052045 | −0.27823 | 0.057876 | −0.54787 | 0.000141 | |
| cg25119415 | MNDA | 0.003595 | −0.2396 | 0.601088 | −0.06934 | 0.002827 | −0.82942 | 0.000144 | |
| cg19149125 | PROSC | 0.007589 | −0.21778 | 0.105621 | −0.20513 | 0.029857 | −0.53916 | 0.000159 | |
| cg10115873 | DNAJB7 | 0.110491 | −0.1285 | 0.003122 | −0.41575 | 0.017411 | −0.64034 | 0.00018 | |
| cg01968178 | REEP1 | 0.006711 | 0.259283 | 0.028211 | 0.325647 | 0.15169 | 0.336464 | 0.000186 | |
| cg22194129 | CLEC4C | 5.46E−06 | −0.39148 | 0.054877 | 0.278152 | 0.004562 | −0.75785 | 0.000194 | |
| cg26240939 | LOC57149 | 0.232468 | −0.09527 | 0.05981 | −0.28894 | 4.81E−05 | −1.28259 | 0.000195 | |
| cg20135306 | SAFB | 0.009706 | −0.22247 | 0.328511 | −0.13256 | 0.004287 | −0.80411 | 0.000195 | |
| cg17886959 | MT2A | 0.012229 | −0.20576 | 0.845246 | −0.02742 | 0.00017 | −1.06956 | 0.000205 | |
| cg13641903 | WT1 | 0.142465 | −0.12177 | 0.016056 | −0.49851 | 0.002232 | −1.20786 | 0.000212 | |
| cg18034329 | RABEP1 | 0.000281 | 0.30783 | 0.185973 | 0.19154 | 0.40452 | 0.204654 | 0.000217 | |
| cg16612562 | RRP22 | 0.037316 | 0.193782 | 0.000277 | 0.612914 | 0.407633 | 0.187779 | 0.000217 | |
| cg10453758 | ACAD11 | 0.124925 | −0.16832 | 0.001093 | −0.57763 | 0.040067 | −0.59109 | 0.000217 | |
| cg17269548 | PPIA | 0.0014 | 0.290685 | 0.352939 | 0.208163 | 0.056514 | 0.717362 | 0.000219 | |
| cg20891917 | IFRD1 | 0.36768 | −0.07899 | 0.018641 | −0.36117 | 8.50E−05 | −1.20042 | 0.000235 | |
| cg07115820 | EPX | 2.42E−05 | −0.34518 | 0.954637 | −0.00958 | 0.244957 | −0.27554 | 0.000248 | |
| cg06542614 | PDLIM1 | 0.002881 | −0.23909 | 0.722625 | −0.04771 | 0.005744 | −0.68835 | 0.000249 | |
| cg01360325 | TAF5 | 0.065308 | 0.167236 | 0.022265 | 0.304756 | 0.011893 | 0.648892 | 0.00025 | |
| cg16399745 | CNAP1 | 0.00828 | −0.22487 | 0.100194 | −0.2478 | 0.052686 | −0.52433 | 0.000256 | |
| cg24505122 | WNT5B | 0.01513 | −0.21316 | 0.009523 | −0.35872 | 0.217755 | −0.30043 | 0.000281 | |
| cg20802392 | CTSK | 0.017821 | −0.21711 | 0.139344 | −0.21339 | 0.013476 | −0.61841 | 0.000284 | |
| cg08805338 | PPP3CB | 0.058493 | 0.16329 | 0.001797 | 0.430975 | 0.132681 | 0.35958 | 0.000286 | |
| cg03535648 | PMCH | 0.000151 | 0.289212 | 0.160246 | 0.201674 | 0.728464 | 0.088116 | 0.000293 | |
| cg03395546 | ADCK4 | 0.008371 | 0.20931 | 0.07953 | 0.33729 | 0.080006 | 0.890978 | 0.000297 | |
| cg22925639 | CHRNA1 | 0.000201 | 0.29074 | 0.523163 | 0.086787 | 0.221173 | 0.335638 | 0.000305 | |
| cg00554173 | ProSAPiP1 | 0.121609 | −0.12872 | 0.020199 | −0.30162 | 0.004867 | −0.678 | 0.000309 | |
| cg21671476 | MYL9 | 0.001138 | −0.30695 | 0.050277 | −0.28487 | 0.578112 | −0.13076 | 0.000316 | |
| cg25514304 | PSEN2 | 0.015902 | −0.20192 | 0.240538 | −0.16615 | 0.007962 | −0.78822 | 0.000321 | |
| cg18397653 | DMP1 | 0.025439 | 0.182345 | 0.030565 | 0.32007 | 0.05939 | 0.492109 | 0.000332 | |
| cg13802966 | CASP1 | 0.000111 | 0.325033 | 0.296412 | 0.161623 | 0.397884 | 0.180387 | 0.000335 | |
| cg02738086 | POLR3H | 0.000234 | −0.32003 | 0.485228 | −0.09631 | 0.247378 | −0.28289 | 0.000344 | |
| cg08023692 | TFDP3 | 0.019678 | 0.267597 | 0.105451 | 0.224376 | 0.023354 | 0.63159 | 0.00035 | |
| cg19061982 | POLR1B | 0.003782 | 0.274987 | 0.194208 | 0.180466 | 0.08423 | 0.471943 | 0.000353 | |
| cg05826823 | CIZ1 | 0.008642 | −0.23058 | 0.042511 | −0.28015 | 0.166221 | −0.36258 | 0.000355 | |
| cg14915165 | WDR3 | 0.000155 | 0.300926 | 0.050418 | 0.257578 | 0.745597 | −0.0782 | 0.000357 | |
| cg27442349 | NFKBIB | 0.002921 | −0.23377 | 0.553159 | −0.08658 | 0.020132 | −0.56082 | 0.000358 | |
| cg06407137 | CD300LB | 0.006972 | −0.21589 | 0.619104 | −0.06901 | 0.004459 | −0.84579 | 0.000363 | |
| cg10710439 | FLJ37549 | 0.001382 | −0.27486 | 0.262314 | −0.16934 | 0.154249 | −0.35187 | 0.000366 | |
| cg21922841 | SLC9A3R1 | 0.001512 | −0.2634 | 0.057328 | −0.27706 | 0.516964 | −0.15463 | 0.000374 | |
| cg11540692 | SIM1 | 0.014593 | 0.232414 | 0.009616 | 0.517408 | 0.302133 | 0.334774 | 0.000393 | |
| cg27210390 | TOM1L1 | 0.022241 | −0.23317 | 0.281152 | −0.21643 | 0.004842 | −0.76416 | 0.000405 | |
| cg23719367 | LONRF1 | 0.000365 | 0.2803 | 0.498478 | 0.093145 | 0.21142 | 0.303544 | 0.000416 | |
| cg05397738 | PGRMC1 | 0.003668 | −0.38198 | 0.306722 | −0.22506 | 0.060928 | −0.72592 | 0.000441 | |
| cg24652919 | WDR58 | 0.029021 | −0.1921 | 0.311465 | −0.13422 | 0.002783 | −0.799 | 0.000445 | |
| cg11231018 | LIPF | 0.002804 | 0.234124 | 0.513497 | 0.087017 | 0.035734 | 0.586248 | 0.000463 | |
| cg04996020 | SLC26A3 | 0.015872 | −0.18898 | 0.049946 | −0.25834 | 0.104261 | −0.39734 | 0.000473 | |

TABLE 5
LnIgE dependent on methylation and cell counts

| probename | pSex | pAge | pMethyl | pParent | pEOS | pNEU | pLYM | pMON | pBAS | pSexAge | pAgeParent | CHR | MAPINFO | SYMBOL | fdrMethyl |
|---|---|---|---|---|---|---|---|---|---|---|---|---|---|---|---|
| cg0199878 | 0.112569 | 0.31381 | 2.36E−06 | 0.127546 | 0.0003 | 0.294806 | 0.300248 | 0.445243 | 0.785135 | 0.561156 | 0.242251 | 16 | 54100210 | AYTL1 | 0.065064 |
| cg1322179 | 0.02397 | 0.509049 | 2.84E−06 | 0.078876 | 1.83E−05 | 0.029037 | 0.081617 | 0.516731 | 0.982861 | 0.487284 | 0.14298 | 13 | 47774920 | RB1 | 0.039182 |
| cg0201710 | 0.024181 | 0.61102 | 2.92E−06 | 0.049837 | 1.36E−11 | 0.071887 | 0.293924 | 0.518605 | 0.971048 | 0.26636 | 0.064733 | 5 | 76824351 | WDR41 | 0.026874 |
| cg1878378 | 0.113216 | 0.331397 | 6.14E−06 | 0.077182 | 0.00065 | 0.866661 | 0.678896 | 0.561016 | 0.551752 | 0.541901 | 0.206229 | 1 | 9521654 | MGC4399 | 0.042316 |
| cg0177040 | 0.195591 | 0.535407 | 6.36E−06 | 0.083123 | 0.000109 | 0.236406 | 0.186757 | 0.667289 | 0.90807 | 0.645592 | 0.182035 | 1 | 1.72E+08 | SERPINC1 | 0.035062 |
| cg2147264 | 0.017156 | 0.280773 | 8.45E−06 | 0.050184 | 3.38E−09 | 0.104506 | 0.231601 | 0.595919 | 0.815002 | 0.229437 | 0.119019 | 7 | 29199993 | CHN2 | 0.038848 |
| cg1644271 | 0.296207 | 0.940445 | 8.64E−06 | 0.011646 | 1.87E−11 | 0.473642 | 0.601444 | 0.132212 | 0.731797 | 0.885131 | 0.036592 | 22 | 38126152 | MAP3K7IP | 0.034039 |
| cg0264366 | 0.383609 | 0.837403 | 9.07E−06 | 0.033857 | 7.18E−06 | 0.384611 | 0.725574 | 0.672431 | 0.976409 | 0.946559 | 0.089254 | 21 | 42659768 | TFF1 | 0.031279 |
| cg1430476 | 0.011159 | 0.942748 | 1.24E−05 | 0.014078 | 1.39E−12 | 0.15707 | 0.53753 | 0.533213 | 0.941455 | 0.331869 | 0.02751 | 9 | 92603970 | SYK | 0.037901 |
| cg2678723 | 0.05919 | 0.499362 | 1.25E−05 | 0.096986 | 1.66E−05 | 0.269226 | 0.259295 | 0.614387 | 0.77939 | 0.282159 | 0.161551 | 5 | 1.32E+08 | IL4 | 0.034585 |
| cg0723638 | 0.034211 | 0.691286 | 1.39E−05 | 0.031758 | 1.14E−11 | 0.448651 | 0.77593 | 0.426761 | 0.862967 | 0.61814 | 0.062142 | 5 | 1.32E+08 | AFF4 | 0.034724 |
| cg0118508 | 0.047791 | 0.954542 | 1.46E−05 | 0.022162 | 2.62E−11 | 0.010595 | 0.092923 | 0.553072 | 0.832145 | 0.287791 | 0.030041 | 15 | 88344817 | ZNF710 | 0.033583 |
| cg2458018 | 0.019297 | 0.776191 | 1.76E−05 | 0.04734 | 9.19E−12 | 0.368871 | 0.807146 | 0.638518 | 0.870483 | 0.542328 | 0.054789 | 2 | 1.6E+08 | WDSUB1 | 0.037432 |
| cg1599694 | 0.168802 | 0.957404 | 3.21E−05 | 0.03806 | 0.000295 | 0.38062 | 0.519495 | 0.662394 | 0.890493 | 0.881234 | 0.065735 | 14 | 49849865 | L2HGDH | 0.063253 |
| cg0011623 | 0.084277 | 0.967515 | 3.87E−05 | 0.045821 | 5.33E−12 | 0.237461 | 0.274079 | 0.360292 | 0.645304 | 0.809802 | 0.047149 | 9 | 18464243 | ADAMTSL1 | 0.071228 |
| cg2565783 | 0.014787 | 0.59541 | 5.67E−05 | 0.07719 | 4.84E−10 | 0.191813 | 0.526463 | 0.689339 | 0.954103 | 0.572083 | 0.149255 | 2 | 11727816 | NTSR2 | 0.097648 |
| cg1860129 | 0.05545 | 0.666807 | 6.08E−05 | 0.079693 | 3.35E−11 | 0.298314 | 0.715483 | 0.403302 | 0.984589 | 0.494763 | 0.111042 | 13 | 78877615 | C13orf10 | 0.098562 |
| cg0140734 | 0.202238 | 0.887784 | 6.20E−05 | 0.042107 | 4.25E−12 | 0.100999 | 0.489071 | 0.205473 | 0.986221 | 0.599695 | 0.074019 | 15 | 95127802 | SPATA8 | 0.095058 |
| cg2039507 | 0.031024 | 0.352683 | 7.03E−05 | 0.038418 | 3.79E−11 | 0.151954 | 0.6381 | 0.523855 | 0.959743 | 0.712082 | 0.097623 | 14 | 52328351 | GNPNAT1 | 0.102077 |
| cg0161475 | 0.034692 | 0.221395 | 7.61E−05 | 0.147953 | 0.000216 | 0.305013 | 0.42021 | 0.601202 | 0.937252 | 0.48583 | 0.296703 | 10 | 44815441 | ZNF22 | 0.104873 |
| cg2549422 | 0.041413 | 0.232534 | 8.31E−05 | 0.079208 | 2.05E−06 | 0.349366 | 0.362883 | 0.456694 | 0.75634 | 0.385361 | 0.208406 | 12 | 10222881 | C12orf59 | 0.109097 |
| cg0358024 | 0.04193 | 0.29297 | 9.19E−05 | 0.05525 | 7.97E−09 | 0.262889 | 0.282218 | 0.522557 | 0.944193 | 0.445805 | 0.168072 | 17 | 39701014 | SLC4A1 | 0.115184 |
| cg1192182 | 0.0119 | 0.406671 | 9.48E−05 | 0.041712 | 2.00E−10 | 0.003869 | 0.022055 | 0.318987 | 0.910154 | 0.284996 | 0.069659 | 1 | 1.56E+08 | FCRL2 | 0.113616 |
| cg0322161 | 0.012405 | 0.545004 | 9.78E−05 | 0.051517 | 7.10E−11 | 0.074755 | 0.312666 | 0.56657 | 0.992653 | 0.361959 | 0.121471 | 19 | 7673348 | FCER2 | 0.112376 |
| cg1015952 | 0.091518 | 0.432871 | 0.000104 | 0.058069 | 6.81E−05 | 0.877227 | 0.802458 | 0.551635 | 0.756105 | 0.58731 | 0.129408 | 3 | 3127530 | IL5RA | 0.114246 |
| cg0379377 | 0 063989 | 0.752523 | 0.000105 | 0.035326 | 3.48E−12 | 0.641755 | 0.759379 | 0.23139 | 0.961909 | 0.535574 | 0.069208 | 19 | 43956560 | LGALS7 | 0.11099 |
| cg0737492 | 0.052443 | 0.618413 | 0.000123 | 0.088611 | 4.32E−11 | 0.340169 | 0.474814 | 0.458963 | 0.837892 | 0.468613 | 0.12446 | 11 | 1.26E+08 | FLJ21103 | 0.125725 |
| cg1867680 | 0.077218 | 0.504848 | 0.000128 | 0.049389 | 1.98E−11 | 0.235957 | 0.646386 | 0.344207 | 0.880866 | 0.660059 | 0.103809 | 3 | 1.71E+03 | MYNN | 0.125972 |
| cg2357819 | 0.016085 | 0.726926 | 0.000135 | 0.03542 | 4.57E−11 | 0.216597 | 0.494102 | 0.527394 | 0.97805 | 0.290345 | 0.064415 | 1 | 2E+08 | LGR6 | 0.128171 |
| cg0094122 | 0.032114 | 0.609516 | 0.000135 | 0.066535 | 8.98E−12 | 0.135225 | 0.585102 | 0.626533 | 0.92613 | 0.387673 | 0.118966 | 1 | 1.48E+08 | ZA20D1 | 0.124366 |
| cg0793603 | 0.069954 | 0.636089 | 0.000137 | 0.038969 | 2.16E−11 | 0.492018 | 0.517777 | 0.254536 | 0.988068 | 0.659643 | 0.066866 | 6 | 7258171 | SSR1 | 0.122262 |
| cg0972501 | 0.098083 | 0.639669 | 0.000139 | 0.06921 | 5.11E−12 | 0.321514 | 0.966281 | 0.6637 | 0.910016 | 0.817038 | 0.084385 | 16 | 27469060 | GTF3C1 | 0.119679 |
| cg2547792 | 0.030102 | 0.496545 | 0.000143 | 0.066909 | 7.47E−12 | 0.458156 | 0.92807 | 0.541779 | 0.878851 | 0.412315 | 0.066247 | 2 | 24437114 | ITSN2 | 0.119115 |
| cg0431082 | 0.054472 | 0.652852 | 0.000147 | 0.053519 | 1.42E−10 | 0.276715 | 0.622541 | 0.517741 | 0.901557 | 0.465279 | 0.08693 | 1 | 53165885 | SCP2 | 0.119353 |
| cg0271216 | 0.044245 | 0.566796 | 0.000167 | 0.051702 | 1.72E−11 | 0.099733 | 0.397373 | 0.352809 | 0.95596 | 0.172671 | 0.084595 | 17 | 16336682 | C17orf76 | 0.131772 |
| cg0660041 | 0.044437 | 0.49994 | 0.000169 | 0.100949 | 2.32E−10 | 0.225498 | 0.838617 | 0.48877 | 0.87749 | 0.581182 | 0.138485 | 4 | 1.86E+08 | MLF1IP | 0.129272 |
| cg1417683 | 0.036603 | 0.521253 | 0.00017 | 0.058407 | 3.20E−12 | 0.358761 | 0.971159 | 0.712563 | 0.899264 | 0.396151 | 0.072729 | 16 | 30391719 | ITGAL | 0.126927 |
| cg0283849 | 0.17373 | 0.742832 | 0.000175 | 0.025634 | 2.84E−11 | 0.10464 | 0.446775 | 0.33717 | 0.942227 | 0.368561 | 0.042349 | 9 | 1.16E+08 | KIF12 | 0.12732 |
| cg2657544 | 0.040069 | 0.670051 | 0.000187 | 0.066446 | 1.63E−10 | 0.250909 | 0.531098 | 0.22331 | 0.884546 | 0.606555 | 0.089471 | 3 | 1.71E+08 | GPR160 | 0.132572 |
| cg2502524 | 0.037574 | 0.258764 | 0.000192 | 0.048148 | 1.44E−11 | 0.31905 | 0.173425 | 0.398194 | 0.777868 | 0.236053 | 0.101869 | 11 | 67106810 | GSTP1 | 0.132572 |
| cg1484942 | 0.026241 | 0.259874 | 0.000218 | 0.096934 | 8.81E−11 | 0.286906 | 0.676816 | 0.282538 | 0.653094 | 0.476361 | 0.258389 | 19 | 62043603 | PEG3 | 0.146738 |
| cg0563789 | 0.12366 | 0.634274 | 0.000232 | 0.04608 | 5.04E−11 | 0.532462 | 0.743939 | 0.22863 | 0.852808 | 0.755366 | 0.070722 | 14 | 30161423 | SCFD1 | 0.152125 |
| cg2234110 | 0.015535 | 0.902518 | 0.000238 | 0.029879 | 1.59E−11 | 0.261008 | 0.392688 | 0.354981 | 0.921805 | 0.422865 | 0.039391 | 1 | 92724849 | GFI1 | 0.152449 |
| cg1032941 | 0.079157 | 0.628662 | 0.000242 | 0.036347 | 2.77E−11 | 0.307235 | 0.35174 | 0.433469 | 0.794075 | 0.444945 | 0.064163 | 7 | 94864117 | PON3 | 0.151626 |
| cg0515439 | 0.031516 | 0.135641 | 0.000245 | 0.102922 | 9.96E−11 | 0.25409 | 0.451921 | 0.439518 | 0.96265 | 0.626335 | 0.354325 | 1 | 36701861 | MRPS15 | 0.15075 |
| cg1716949 | 0.029075 | 0.563407 | 0.000252 | 0.042441 | 6.59E−12 | 0.17547 | 0.322044 | 0.3052 | 0.7201 | 0.580706 | 0.084037 | 1 | 11663252 | MAD2L2 | 0.151029 |
| cg1618120 | 0.067764 | 0.591989 | 0.000252 | 0.042956 | 7.27E−12 | 0.378764 | 0.960736 | 0.653767 | 0.955953 | 0.540424 | 0.054842 | 4 | 1.57E+08 | CTSO | 0.147901 |
| cg1651829 | 0.046342 | 0.496269 | 0.000253 | 0.04826 | 1.67E−11 | 0.490558 | 0.882164 | 0.686869 | 0.969441 | 0.555757 | 0.054603 | 19 | 62484069 | ZNF272 | 0.14527 |
| cg2508271 | 0.0424 | 0.636757 | 0.000256 | 0.020147 | 1.06E−11 | 0.072407 | 0.390426 | 0.358877 | 0.988008 | 0.593926 | 0.023695 | 1 | 1.51E+08 | IVL | 0.143934 |
| cg0662665 | 0.095183 | 0.533265 | 0.000283 | 0.034735 | 7.19E−12 | 0.470461 | 0.894785 | 0.348132 | 0.884842 | 0.492447 | 0.062698 | 1 | 37872503 | RSPO1 | 0.155898 |
| cg1672979 | 0.04751 | 0.601101 | 0.000287 | 0.116642 | 5.94E−12 | 0.137858 | 0.594131 | 0.368461 | 0.622713 | 0.343005 | 0.132747 | 3 | 39484199 | MOBP | 0.155157 |
| cg0066148 | 0.049675 | 0.956779 | 0.00029 | 0.034644 | 1.39E−10 | 0.051415 | 0.363871 | 0.94523 | 0.887957 | 0.732528 | 0.055126 | 5 | 1.69E+08 | FOXI1 | 0.153684 |
| cg2595796 | 0.079067 | 0.876029 | 0.000297 | 0.02787 | 7.28E−12 | 0.385781 | 0.943596 | 0.555259 | 0.89036 | 0.824112 | 0.047475 | 1 | 55277736 | PCSK9 | 0.154427 |
| cg2582069 | 0.010244 | 0.583386 | 0.000306 | 0.047556 | 1.21E−13 | 0.341316 | 0.838957 | 0.536776 | 0.778258 | 0.446869 | 0.064677 | 1 | 1.49E+08 | SEMA6C | 0.156114 |
| cg0327165 | 0.077777 | 0.712104 | 0.000337 | 0.038887 | 2.67E−10 | 0.418453 | 0.487654 | 0.311084 | 0.75272 | 0.65733 | 0.105987 | 1 | 1.5E+08 | POGZ | 0.168849 |
| cg2148665 | 0.045486 | 0.40522 | 0.000358 | 0.070729 | 5.38E−12 | 0.284056 | 0.70665 | 0.636245 | 0.917597 | 0.385696 | 0.123213 | 6 | 10855679 | TMEM14B | 0.176112 |
| cg2167147 | 0.046581 | 0.413423 | 0.000361 | 0.055718 | 2.78E−09 | 0.026938 | 0.088417 | 0.528128 | 0.839607 | 0.448265 | 0.100735 | 20 | 34603023 | MYL9 | 0.17447 |
| cg2026953 | 0.026881 | 0.508034 | 0.000373 | 0.026582 | 3.31E−11 | 0.100635 | 0.326035 | 0.4596 | 0.976638 | 0.356229 | 0.0502 | 22 | 44445738 | ATXN10 | 0.177283 |
| cg0369309 | 0.098715 | 0.427746 | 0.000383 | 0.064916 | 9.27E−06 | 0.099207 | 0.18224 | 0.575481 | 0.93957 | 0.524595 | 0.113142 | 9 | 1.35E+08 | CEL | 0.179022 |
| cg1171809 | 0.021019 | 0.423372 | 0.000394 | 0.049605 | 5.99E−11 | 0.146104 | 0.434936 | 0.288398 | 0.665145 | 0.582588 | 0.087605 | 1 | 1.44E+08 | NOTCH2NL | 0.181095 |
| cg1223246 | 0.084846 | 0.590253 | 0.000396 | 0.038592 | 1.55E−10 | 0.131274 | 0.18585 | 0.363367 | 0.913712 | 0.682457 | 0.034866 | 2 | 1E+08 | LONRF2 | 0.178997 |
| cg1262924 | 0.390357 | 0.801426 | 0.000398 | 0.0121 | 2.26E−12 | 0.226708 | 0.525541 | 0.233808 | 0.969579 | 0.998939 | 0.022438 | 1 | 1.77E+08 | FAM208 | 0.176812 |
| cg1912532 | 0.027654 | 0.521308 | 0.000399 | 0.045215 | 9.57E−12 | 0.255824 | 0.647856 | 0.224689 | 0.893996 | 0.493623 | 0.078119 | 17 | 72245065 | SFRS2 | 0.174528 |
| cg2463195 | 0.040808 | 0.712642 | 0.000412 | 0.046456 | 3.23E−11 | 0.222001 | 0.639819 | 0.540534 | 0.818026 | 0.481945 | 0.07343 | 10 | 59764631 | UBE2D1 | 0.177376 |
| cg2624613 | 0.430557 | 0.77411 | 0.000415 | 0.02223 | 1.51E−11 | 0.318576 | 0.587407 | 0.30291 | 0.64211 | 0.892287 | 0.030011 | X | 18282533 | SCML2 | 0.17608 |
| cg0283887 | 0.036979 | 0.380853 | 0.000418 | 0.062458 | 7.07E−11 | 0.357586 | 0.462406 | 0.254022 | 0.826859 | 0.522711 | 0.107281 | 13 | 96671878 | MBNL2 | 0.174819 |
| cg0224158 | 0.091123 | 0.689763 | 0.000448 | 0.044858 | 1.10E−11 | 0.408221 | 0.8686 | 0.534983 | 0.970304 | 0.629906 | 0.068962 | 2 | 2.09E+08 | PIP5K3 | 0.184404 |
| cg0256961 | 0.040718 | 0.840021 | 0.000455 | 0.061807 | 1.55E−10 | 0.028409 | 0.088431 | 0.46958 | 0.924587 | 0.734897 | 0.092383 | 10 | 49993133 | C10orf72 | 0.184379 |
| cg0293216 | 0.212145 | 0.893422 | 0.00047 | 0.015619 | 3.99E−12 | 0.229375 | 0.933099 | 0.681222 | 0.920634 | 0.500792 | 0.029959 | 2 | 2.33E+08 | ECEL1 | 0.18779 |
| cg0967639 | 0.098285 | 0.766752 | 0.000472 | 0.030336 | 3.48E−07 | 0.432091 | 0.530921 | 0.462608 | 0.820656 | 0.618912 | 0.064331 | 21 | 45317773 | ADARB1 | 0.186116 |

TABLE 6
Independent predictors of total serum IgE concentration in MRCA panel

| | | | | | | V CGI | V CGI |
|---|---|---|---|---|---|---|---|
| Probe | Gene | P for step | V Sex | V Age | V EOS | total | step |
| cg18783781 | SLC25A33 | 2.2 × 10 − 6 | 1.0% | 10.4% | 12.1% | 8.5% | 8.5% |
| cg01998785 | LPCAT2 | 5.4 × 10 − 3 | 1.0% | 11.6% | 9.9% | 11.5% | 3.0% |
| cg15996947 | L2HGDH | 7.0 × 10 − 2 | 1.0% | 11.3% | 8.8% | 13.7% | 2.2% |

The results of a stepwise regression are shown with estimates of variation (V) for significant covariates. LnIgE is the dependent variable. The models included all CGI loci with genome-wide significant association to LnIgE, age, sex, parental status, and eosinophil, neutrophil, lymphocyte, monocyte and basophil counts.

Markers are identified through their Illumina IDs and the associated Gene symbol is derived from the IIlumina annotation updated through PubMed.

TABLE 7
Comparison of surrogate variable analyses with direct white cell counts in association models

| | a) Before adjusting cell counts | | | | b) Adjusting for Houseman cell proportions | | | | | | | c) Adjusting for white cell counts | | | | | |
|---|---|---|---|---|---|---|---|---|---|---|---|---|---|---|---|---|---|
| Chr. | Position | Symbol | probe | pMethy | pMethy | pCD8T | pCD4T | pNK | pBCELL | pMON | pGRAN | pMethy | pEOS | pNEU | pLYM | pMON | pBAS |
| 1 | 9521654 | SLC25A33 | cg18783781 | 4.96E−14 | 5.58E−13 | 0.3625663 | 0.6489145 | 0.0830001 | 0.0735391 | 0.7715233 | 0.2564499 | 6.14E−06 | 0.00065 | 0.866661 | 0.678896 | 0.561016 | 0.551752 |
| 16 | 54100210 | LPCAT2 | cg01998785 | 1.24E−13 | 3.81E−13 | 0.1560292 | 0.3633042 | 0.034881 | 0.0300831 | 0.7239839 | 0.1881862 | 2.36E−06 | 0.0003 | 0.294806 | 0.300248 | 0.445243 | 0.785135 |
| 14 | 49849865 | L2HGDH | cg15996947 | 7.39E−13 | 3.81E−11 | 0.3709841 | 0.6823007 | 0.125117 | 0.1583239 | 0.9387673 | 0.3768824 | 3.21E−05 | 0.000295 | 0.38062 | 0.519495 | 0.662394 | 0.890493 |
| 10 | 44815441 | ZNF22 | cg01614759 | 4.42E−12 | 9.09E−11 | 0.3526504 | 0.6420391 | 0.1382946 | 0.0810812 | 0.654088 | 0.3845248 | 7.61E−05 | 0.000216 | 0.305013 | 0.42021 | 0.601202 | 0.937252 |
| 3 | 3127530 | IL5RA | cg10159529 | 5.06E−12 | 1.70E−10 | 0.8786628 | 0.9388517 | 0.2941882 | 0.2701973 | 0.9752202 | 0.5129874 | 0.000104 | 6.81E−05 | 0.877227 | 0.802458 | 0.551635 | 0.756105 |
| 1 | 172153108 | SERPINC1 | cg01770400 | 6.59E−12 | 1.54E−11 | 0.1182883 | 0.3063038 | 0.0323251 | 0.0368702 | 0.574556 | 0.1896 | 6.36E−06 | 0.000109 | 0.236406 | 0.186757 | 0.667289 | 0.90807 |
| 21 | 42659768 | TFF1 | cg02643667 | 7.87E−12 | 6.81E−11 | 0.234861 | 0.4646176 | 0.0330799 | 0.0706689 | 0.797703 | 0.1753888 | 9.07E−06 | 7.18E−06 | 0.384611 | 0.725574 | 0.672431 | 0.976409 |
| 5 | 132036424 | IL4 | cg26787239 | 1.59E−11 | 2.13E−10 | 0.5058003 | 0.9922092 | 0.1723318 | 0.1996409 | 0.7364825 | 0.6544331 | 1.25E−05 | 1.66E−05 | 0.269226 | 0.259295 | 0.614387 | 0.77939 |
| 2 | 110014086 | LIMS3 | cg18879041 | 4.06E−11 | 2.57E−09 | 0.3708111 | 0.7030526 | 0.1613161 | 0.2166898 | 0.9745824 | 0.474026 | 0.000865 | 0.000256 | 0.280498 | 0.382954 | 0.500203 | 0.963467 |
| 13 | 47774920 | RB1 | cg13221796 | 5.66E−10 | 9.26E−11 | 0.1278218 | 0.3641314 | 0.0353766 | 0.0458363 | 0.7786051 | 0.2987393 | 2.84E−06 | 1.83E−05 | 0.029037 | 0.081617 | 0.516731 | 0.982861 |
| 19 | 60432000 | TMEM86B | cg26457013 | 1.02E−09 | 4.08E−09 | 0.3305229 | 0.5920938 | 0.036091 | 0.0792481 | 0.9078355 | 0.2711535 | 0.000691 | 1.35E−05 | 0.363948 | 0.317448 | 0.558421 | 0.740734 |
| 9 | 100745613 | COL15A1 | cg20503329 | 1.19E−09 | 3.14E−08 | 0.4772819 | 0.7388277 | 0.1359031 | 0.1343368 | 0.9433195 | 0.4284566 | 0.001341 | 1.17E−05 | 0.443838 | 0.49736 | 0.401174 | 0.959364 |
| 1 | 31166502 | SDC3 | cg07689731 | 2.33E−09 | 4.79E−09 | 0.5074834 | 0.7985815 | 0.0774502 | 0.0726484 | 0.7924958 | 0.4108301 | 0.002906 | 1.48E−05 | 0.415182 | 0.483806 | 0.437372 | 0.932638 |
| 21 | 45317773 | ADARB1 | cg09676390 | 1.21E−08 | 1.32E−07 | 0.43369 | 0.6702876 | 0.0791369 | 0.1103603 | 0.9234331 | 0.3292778 | 0.000472 | 3.48E−07 | 0.432091 | 0.530921 | 0.462608 | 0.820656 |
| 9 | 134926722 | CEL | cg03693099 | 1.84E−08 | 1.01E−07 | 0.2926779 | 0.512945 | 0.0818301 | 0.1071869 | 0.9754856 | 0.3848947 | 0.000383 | 9.27E−06 | 0.099207 | 0.18224 | 0.575481 | 0.93957 |
| 19 | 12859426 | KLF1 | cg26136776 | 3.34E−08 | 5.42E−07 | 0.2328607 | 0.4764967 | 0.0498737 | 0.087521 | 0.7661611 | 0.2312155 | 0.008494 | 4.25E−06 | 0.308908 | 0.378613 | 0.526765 | 0.764392 |
| X | 48529968 | GATA1 | cg00536175 | 7.85E−08 | 5.70E−07 | 0.6056632 | 0.9159691 | 0.163687 | 0.1102612 | 0.8082841 | 0.5675393 | 0.002444 | 1.31E−06 | 0.230592 | 0.333849 | 0.423511 | 0.745501 |
| 12 | 10222881 | TMEM52B | cg25494227 | 1.29E−07 | 2.79E−07 | 0.4810187 | 0.6746518 | 0.0457622 | 0.0735827 | 0.9017706 | 0.3386288 | 8.31E−05 | 2.05E−08 | 0.349366 | 0.362883 | 0.456694 | 0.75634 |
| 9 | 33229641 | SPINK4 | cg00079056 | 1.54E−07 | 1.23E−06 | 0.3893041 | 0.6595419 | 0.067607 | 0.090288 | 0.950103 | 0.3262354 | 0.015058 | 9.70E−07 | 0.327361 | 0.481372 | 0.49343 | 0.779557 |
| 12 | 15017287 | PDE6H | cg09447105 | 2.16E−07 | 1.80E−06 | 0.6038815 | 0.9752817 | 0.1325361 | 0.2619219 | 0.6725442 | 0.6818338 | 0.002327 | 6.73E−07 | 0.185319 | 0.308805 | 0.650588 | 0.72691 |
| 16 | 4778723 | FLJ25410 | cg05215575 | 3.11E−07 | 7.40E−08 | 0.6354446 | 0.6693211 | 0.0382164 | 0.0635217 | 0.8705757 | 0.2084314 | 0.011151 | 9.08E−08 | 0.699883 | 0.891282 | 0.341595 | 0.856081 |
| 7 | 142369547 | KEL | cg17784922 | 4.24E−07 | 9.07E−07 | 0.6926023 | 0.9854571 | 0.0699431 | 0.1577229 | 0.8028496 | 0.5052827 | 0.001391 | 3.21E−08 | 0.505128 | 0.698535 | 0.367309 | 0.99912 |
| 7 | 29199993 | CHN2 | cg21472642 | 6.51E−07 | 7.39E−07 | 0.5225896 | 0.8312874 | 0.0708089 | 0.0764603 | 0.9022296 | 0.4725488 | 8.45E−06 | 3.38E−09 | 0.104506 | 0.231601 | 0.595919 | 0.815002 |
| 2 | 191009029 | FLJ20160 | cg15998761 | 9.33E−07 | 9.09E−06 | 0.4236314 | 0.824669 | 0.1393183 | 0.1145142 | 0.8527446 | 0.4643437 | 0.043732 | 5.71E−07 | 0.331558 | 0.449501 | 0.418024 | 0.785659 |
| 6 | 25862169 | SLC17A4 | cg21627181 | 1.14E−06 | 1.42E−06 | 0.7663137 | 0.9149809 | 0.0591244 | 0.1414494 | 0.9511792 | 0.3972344 | 0.025202 | 5.30E−08 | 0.609342 | 0.865393 | 0.467443 | 0.746032 |
| 14 | 49849874 | L2HGDH | cg20189937 | 1.32E−06 | 1.16E−05 | 0.503935 | 0.9204654 | 0.0921306 | 0.1950088 | 0.8817908 | 0.5174749 | 0.007269 | 1.26E−07 | 0.297519 | 0.525586 | 0.480584 | 0.958405 |
| 17 | 39822093 | ITGA2B | cg17749520 | 1.37E−06 | 9.24E−06 | 0.3677214 | 0.6752543 | 0.046072 | 0.1157269 | 0.8717913 | 0.3274826 | 0.009625 | 3.11E−07 | 0.286277 | 0.406727 | 0.666483 | 0.903995 |
| 17 | 39701014 | SLC4A1 | cg03580247 | 1.51E−06 | 1.76E−05 | 0.4966594 | 0.7030166 | 0.1566437 | 0.1201467 | 0.7706213 | 0.4809553 | 9.19E−05 | 7.97E−09 | 0.262889 | 0.282218 | 0.522557 | 0.944193 |
| 20 | 41789039 | FAM112A | cg11398517 | 2.42E−06 | 2.11E−05 | 0.431888 | 0.7316457 | 0.0506171 | 0.209415 | 0.8337169 | 0.4256838 | 0.006763 | 1.86E−07 | 0.220078 | 0.444385 | 0.592602 | 0.918166 |
| 11 | 56904791 | PRG3 | cg24459209 | 3.33E−06 | 1.29E−05 | 0.5460024 | 0.9366639 | 0.0692143 | 0.1545826 | 0.8947187 | 0.4338765 | 0.094895 | 2.69E−07 | 0.444762 | 0.62114 | 0.500854 | 0.993962 |
| 12 | 7792928 | CLEC4C | cg22194129 | 5.46E−06 | 0.0001208 | 0.4977092 | 0.8378959 | 0.0710935 | 0.6019842 | 0.8886589 | 0.4447889 | 0.023532 | 2.38E−08 | 0.408481 | 0.759002 | 0.521453 | 0.818552 |
| 14 | 95011802 | C14orf49 | cg16522484 | 8.04E−06 | 0.0001668 | 0.4308412 | 0.7282314 | 0.1486239 | 0.1112817 | 0.9482318 | 0.4206034 | 0.034072 | 1.01E−07 | 0.285777 | 0.449644 | 0.49125 | 0.837548 |
| X | 48529554 | GATA1 | cg22543648 | 9.51E−06 | 7.12E−05 | 0.682124 | 0.9511741 | 0.1312401 | 0.1917101 | 0.6997158 | 0.4748147 | 0.02664 | 2.00E−08 | 0.585973 | 0.689124 | 0.425854 | 0.888172 |
| 2 | 11727816 | NTSB2 | cg25657834 | 1.02E−05 | 2.04E−05 | 0.9354409 | 0.6331433 | 0.1373482 | 0.3261244 | 0.4920064 | 0.8015518 | 5.67E−05 | 4.84E−10 | 0.191813 | 0.526463 | 0.689339 | 0.954103 |
| 19 | 48912054 | IRGC | cg26251865 | 1.76E−05 | 9.19E−06 | 0.714389 | 0.9562959 | 0.0555475 | 0.1095196 | 0.6911884 | 0.4367507 | 0.011533 | 1.35E−08 | 0.495745 | 0.567965 | 0.468957 | 0.977824 |
| 19 | 44919789 | CLC | cg18254848 | 1.77E−05 | 2.33E−05 | 0.7122613 | 0.874791 | 0.0834931 | 0.1954486 | 0.6341788 | 0.5277066 | 0.161753 | 4.77E−08 | 0.462599 | 0.67872 | 0.424877 | 0.937408 |
| 6 | 10635801 | GCNT2 | cg26385286 | 1.78E−05 | 3.88E−05 | 0.5747779 | 0.9758809 | 0.0654066 | 0.2046998 | 0.6574459 | 0.5308099 | 0.070861 | 4.31E−08 | 0.339931 | 0.593173 | 0.451377 | 0.902264 |
| 3 | 150064527 | CPA3 | cg13424229 | 1.89E−05 | 2.29E−06 | 0.4329161 | 0.6962458 | 0.1048729 | 0.0674688 | 0.7084296 | 0.6214492 | 0.001236 | 4.96E−07 | 0.054043 | 0.094343 | 0.451245 | 0.955257 |

Table 7 shows the I values from regression models in the MRCA panel that predict InIgE at each locus a) before adjusting cell counts; b) after adjusting cell counts using Houseman surrogate variables; c) after adjusting for cell subsets counted in our data. pMethyl measures strength of association to IgE. CD8, CD4 and NK are T cell subsets, GRAN=granulocytes, EOS=eosinophils, NEU=neutrophils, LYM=lymphocytes, MON=monocytes, BAS=basophils.

All models were adjusted for sex, age, methylation, parent/child status, sex*age interaction, and age*parent interaction.

TABLE 8
Ln(IgE) associations with CGI in three populations with meta-analysis and in isolated eosinophils
Association results are shown for the MRCA, SLSJ and PAPA panels of subjects, together with the meta-analysis.
P = P value, e = effect size, FDR = false discovery rate, P ex vivo = P values from isolated eosinophils (shown in FIG. 3).

| Probe | Symbol | P MRCA | e MRCA | P SLSJ | e SLSJ |
|---|---|---|---|---|---|
| cg01998785 | LPCAT2 | 1.24E−13 | −0.59568648 | 0.00802557 | −0.40092223 |
| cg10159529 | IL5RA | 5.06E−12 | −0.5617836 | 0.0002081 | −0.55179883 |
| cg01614759 | ZNF22 | 4.42E−12 | −0.55724314 | 0.00344386 | −0.41241316 |
| cg15996947 | L2HGDH | 7.39E−13 | −0.60591889 | 0.01032468 | −0.36549518 |
| cg26787239 | IL4 | 1.59E−11 | −0.56340788 | 0.00126475 | −0.43653677 |
| cg18783781 | SLC25A33 | 4.96E−14 | −0.62715723 | 0.03433049 | −0.30221877 |
| cg13221796 | RB1 | 5.66E−10 | −0.50354586 | 0.05961919 | −0.25179816 |
| cg01770400 | SERPINC1 | 6.59E−12 | −0.55737559 | 0.00695942 | −0.37241711 |
| cg02643667 | TFF1 | 7.87E−12 | −0.59305233 | 0.13160859 | −0.21305788 |
| cg21627181 | SLC17A4 | 1.14E−06 | −0.39818005 | 0.00013788 | −0.57100223 |
| cg20189937 | L2HGDH | 1.32E−06 | −0.38816073 | 0.00236714 | −0.41628707 |
| cg26457013 | TMEM86B | 1.02E−09 | −0.49167438 | 0.09531643 | −0.23285921 |
| cg20503329 | COL15A1 | 1.19E−09 | −0.48099203 | 0.30013505 | −0.13390743 |
| cg03693099 | CEL | 1.84E−08 | −0.4748768 | 0.10864305 | −0.21581211 |
| cg00079056 | SPINK4 | 1.54E−07 | −0.42210571 | 0.06391069 | −0.26671943 |
| cg09676390 | ADARB1 | 1.21E−08 | −0.49309876 | 0.07017494 | −0.26519869 |
| cg15998761 | FLJ20160 | 9.33E−07 | −0.39912034 | 0.01317172 | −0.3493404 |
| cg25494227 | C12orf59 | 1.29E−07 | −0.44273395 | 0.19908916 | −0.17654518 |
| cg11398517 | FAM112A | 2.42E−06 | −0.38183485 | 0.00740442 | −0.43205317 |
| cg06690548 | SLC7A11 | 2.75E−05 | −0.34239536 | 0.00033024 | −0.5124057 |
| cg17784922 | KEL | 4.24E−07 | −0.39654946 | 0.00788237 | −0.36643857 |
| cg16050349 | PIK3CB | 4.03E−05 | −0.33563755 | 0.00174335 | −0.43168912 |
| cg25636075 | TMEM41A | 0.00025173 | −0.2971522 | 5.06E−05 | −0.58390318 |
| cg08404225 | IL5RA | 0.00022963 | −0.30480089 | 0.00327113 | −0.42850193 |
| cg09447105 | PDE6H | 2.16E−07 | −0.41885464 | 0.13026736 | −0.23288626 |
| cg05215575 | FLJ25410 | 3.11E−07 | −0.43075634 | 0.21027916 | −0.18075898 |
| cg26136776 | KLF1 | 3.34E−08 | −0.44803079 | 0.42956532 | −0.10677731 |
| cg17749520 | ITGA2B | 1.37E−06 | −0.429428 | 0.02472277 | −0.33917361 |
| cg24459209 | PRG3 | 3.33E−06 | −0.38833512 | 0.02944954 | −0.31500414 |
| cg00002426 | SLMAP | 7.92E−05 | −0.32642478 | 0.00832614 | −0.3856364 |
| cg15357945 | PRG2 | 0.00221404 | −0.28738419 | 2.82E−05 | −0.60297106 |
| cg17582777 | EFNA3 | 0.00010748 | −0.32224372 | 0.03115439 | −0.29044811 |
| cg19881895 | SLC43A3 | 7.50E−05 | −0.31885674 | 0.00278684 | −0.40198707 |
| cg18254848 | CLC | 1.77E−05 | −0.34919529 | 0.04441021 | −0.28497126 |
| cg21631409 | ALDH3B2 | 0.00023412 | −0.29131847 | 0.01689366 | −0.32960636 |
| cg00536175 | GATA1 | 7.85E−08 | −0.4363586 | 0.40165516 | −0.14261506 |
| cg04111761 | CCR3 | 0.00011711 | −0.33370177 | 0.23351172 | −0.17447541 |
| cg16386158 | IL1RL1 | 0.00101563 | −0.26235478 | 0.0659533 | −0.27398766 |
| cg12866859 | HEXIM1 | 4.09E−05 | −0.33394077 | 0.02976428 | −0.31862609 |
| cg26251865 | IRGC | 1.76E−05 | −0.35144075 | 0.22190136 | −0.16501014 |
| cg17890764 | ITIH4 | 0.00186295 | −0.27823155 | 0.0684601 | −0.27450935 |
| cg16522484 | C14orf49 | 8.04E−06 | −0.35880421 | 0.44941893 | −0.11510558 |
| cg08377000 | TIGD2 | 0.00024363 | −0.30373794 | 0.08093135 | −0.23266769 |
| cg13424229 | CPA3 | 1.89E−05 | −0.38251413 | 0.08348589 | −0.23336581 |
| cg26385286 | GCNT2 | 1.78E−05 | −0.34608075 | 0.87728926 | −0.02144509 |
| cg10805676 | MRPL28 | 3.73E−05 | −0.35955303 | 0.30508361 | −0.1427004 |
| cg27653134 | A2ML1 | 0.00242961 | −0.23973051 | 0.22905657 | −0.17252069 |
| cg10414946 | MS4A2 | 0.01049471 | −0.23378663 | 0.00575798 | −0.43389042 |
| cg10280342 | PSPN | 0.00039749 | −0.28368918 | 0.02629919 | −0.32857148 |
| cg16396488 | PLA2G1B | 0.00017825 | −0.30651705 | 0.39238082 | −0.12128199 |
| cg23759710 | OXER1 | 0.0001787 | −0.30704267 | 0.25364966 | −0.16120224 |
| cg07689731 | SDC3 | 2.33E−09 | −0.48818944 | 0.28473894 | −0.14857293 |
| cg09793866 | STAR | 0.04748074 | −0.16691732 | 0.01351705 | −0.3256221 |
| cg06736444 | SRRM2 | 0.00054411 | −0.29121824 | 0.06268254 | −0.25598132 |
| cg20967028 | ART4 | 0.0005779 | −0.28834882 | 0.17073372 | −0.18058886 |
| cg21682902 | HAL | 0.00872319 | −0.22446114 | 0.03781436 | −0.29880795 |
| cg04523589 | CAMP | 0.00107516 | −0.28064177 | 0.11458551 | −0.21991497 |
| cg03014680 | CLEC12A | 0.00011061 | −0.3170943 | 0.17486517 | −0.18646312 |
| cg23064554 | CTRC | 0.00192856 | −0.25292356 | 0.04939187 | −0.24890418 |
| cg00596686 | STS | 0.01311846 | −0.22173209 | 0.04966498 | −0.35212463 |
| cg07374928 | FLJ21103 | 0.00019885 | 0.28824296 | 0.04532997 | 0.26918523 |
| cg03580247 | SLC4A1 | 1.51E−06 | −0.40249841 | 0.5798195 | −0.073759 |
| cg06394229 | LGALS4 | 0.03922521 | −0.17088433 | 0.00698054 | −0.36506666 |
| cg22543648 | GATA1 | 9.51E−06 | −0.46480889 | 0.81678324 | −0.0313351 |
| cg05154390 | MRPS15 | 0.00024707 | −0.36152271 | 0.13371749 | −0.26479294 |
| cg12818699 | C6orf32 | 0.00213296 | −0.28352411 | 0.23042981 | −0.1896684 |
| cg05869585 | PM M2 | 0.00757356 | −0.21216585 | 0.33559928 | −0.13831414 |
| cg11136251 | ZWILCH | 0.00275583 | 0.24936479 | 0.04553809 | 0.25521894 |
| cg09914444 | DMBX1 | 0.01051229 | −0.23967264 | 0.00364804 | −0.4455015 |
| Probe | P PAPA | e PAPA | P Meta | metaFDR | P ex vivo |
| cg01998785 | 9.60E−06 | −1.46270087 | 1.16E−18 | 2.92E−14 | 2.18E−02 |
| cg10159529 | 7.25E−05 | −1.33591029 | 2.17E−18 | 2.73E−14 | 1.26E−02 |
| cg01614759 | 2.81E−06 | −1.79230787 | 2.83E−18 | 2.37E−14 | 1.77E−02 |
| cg15996947 | 3.73E−05 | −1.20483794 | 2.75E−17 | 1.73E−13 | 1.57E−02 |
| cg26787239 | 0.0004795 | −1.06347991 | 3.24E−16 | 1.63E−12 | 8.75E−03 |
| cg18783781 | 0.00618644 | −0.68377782 | 4.43E−15 | 1.86E−11 | 8.79E−03 |
| cg13221796 | 4.95E−06 | −1.69670252 | 2.49E−14 | 8.92E−11 | 5.37E−02 |
| cg01770400 | 0.01263081 | −0.63026886 | 5.27E−14 | 1.65E−10 | 9.76E−03 |
| cg02643667 | 0.00259602 | −0.9862388 | 7.59E−13 | 2.12E−09 | 6.99E−05 |
| cg21627181 | 0.00037841 | −1.03867051 | 1.13E−12 | 2.85E−09 | 1.57E−02 |
| cg20189937 | 3.19E−05 | −1.68808338 | 2.55E−12 | 5.83E−09 | 1.52E−02 |
| cg26457013 | 0.0009001 | −0.89216051 | 7.06E−12 | 1.48E−08 | 6.73E−03 |
| cg20503329 | 8.83E−05 | −1.24103621 | 9.50E−12 | 1.84E−08 | 3.73E−03 |
| cg03693099 | 8.30E−05 | −1.35571957 | 1.32E−11 | 2.36E−08 | 8.24E−03 |
| cg00079056 | 3.13E−05 | −1.3642948 | 1.81E−11 | 3.03E−08 | 2.69E−03 |
| cg09676390 | 0.00080324 | −0.90872655 | 3.06E−11 | 4.81E−08 | 8.48E−03 |
| cg15998761 | 0.000171 | −1.18429241 | 4.55E−11 | 6.72E−08 | 1.25E−02 |
| cg25494227 | 1.18E−05 | −1.47126377 | 5.09E−11 | 7.10E−08 | 8.45E−03 |
| cg11398517 | 0.00032805 | −1.06414046 | 1.02E−10 | 1.34E−07 | 3.06E−03 |
| cg06690548 | 0.00106969 | −0.87874451 | 1.79E−10 | 2.24E−07 | 2.26E−02 |
| cg17784922 | 0.00440294 | −0.72454617 | 2.14E−10 | 2.56E−07 | 2.71E−03 |
| cg16050349 | 0.00022545 | −1.01162458 | 3.23E−10 | 3.69E−07 | 9.01E−03 |
| cg25636075 | 0.00077233 | −1.01625779 | 3.86E−10 | 4.22E−07 | 5.55E−03 |
| cg08404225 | 8.41E−06 | −1.70975411 | 4.11E−10 | 4.30E−07 | 1.12E−02 |
| cg09447105 | 0.00040358 | −1.04935027 | 5.27E−10 | 5.30E−07 | 6.44E−03 |
| cg05215575 | 0.00027239 | −1.16021304 | 1.19E−09 | 1.15E−06 | |
| cg26136776 | 0.00066101 | −0.9590904 | 1.54E−09 | 1.44E−06 | |
| cg17749520 | 0.00346624 | −0.84297101 | 1.76E−09 | 1.58E−06 | |
| cg24459209 | 0.00110489 | −0.85001483 | 1.81E−09 | 1.57E−06 | |
| cg00002426 | 0.00016162 | −1.20424971 | 2.38E−09 | 1.99E−06 | |
| cg15357945 | 0.0005806 | −0.94556968 | 3.14E−09 | 2.55E−06 | |
| cg17582777 | 8.22E−05 | −1.26150193 | 8.56E−09 | 6.72E−06 | |
| cg19881895 | 0.00673483 | −0.68559874 | 1.63E−08 | 1.24E−05 | |
| cg18254848 | 0.00456599 | −0.72531777 | 4.52E−08 | 3.34E−05 | |
| cg21631409 | 0.00122328 | −0.86863093 | 6.83E−08 | 4.90E−05 | |
| cg00536175 | 0.05059656 | −0.55841279 | 1.41E−07 | 9.85E−05 | |
| cg04111761 | 0.00016201 | −1.26197396 | 2.10E−07 | 0.00014261 | |
| cg16386158 | 0.00014816 | −1.08979267 | 3.31E−07 | 0.00021888 | |
| cg12866859 | 0.03024259 | −0.60307739 | 3.54E−07 | 0.0002279 | |
| cg26251865 | 0.00497396 | −0.79575908 | 3.97E−07 | 0.00024959 | |
| cg17890764 | 9.81E−05 | −1.07966704 | 5.25E−07 | 0.00032184 | |
| cg16522484 | 0.00738786 | −0.72584311 | 9.34E−07 | 0.00055892 | |
| cg08377000 | 0.00433821 | −0.76763882 | 1.00E−06 | 0.00058496 | |
| cg13424229 | 0.06845531 | −0.5217078 | 1.30E−06 | 0.00074281 | |
| cg26385286 | 0.00064614 | −0.92037966 | 1.36E−06 | 0.00075804 | |
| cg10805676 | 0.00846068 | −0.60542678 | 1.87E−06 | 0.001024 | |
| cg27653134 | 4.21E−05 | −1.33609912 | 2.03E−06 | 0.00108581 | |
| cg10414946 | 0.001316 | −0.93259583 | 2.04E−06 | 0.00106538 | |
| cg10280342 | 0.04176091 | −0.55637584 | 3.41E−06 | 0.00174862 | |
| cg16396488 | 0.0018756 | −0.84923218 | 3.58E−06 | 0.00179677 | |
| cg23759710 | 0.00628062 | −0.74809459 | 4.31E−06 | 0.00212414 | |
| cg07689731 | 0.49681968 | 0.18110297 | 4.97E−06 | 0.00240015 | |
| cg09793866 | 7.28E−05 | −1.17221264 | 5.53E−06 | 0.00262059 | |
| cg06736444 | 0.02479561 | −0.60662331 | 6.62E−06 | 0.00307895 | |
| cg20967028 | 0.00747774 | −0.67519402 | 8.31E−06 | 0.00379777 | |
| cg21682902 | 0.00137829 | −0.93095454 | 8.41E−06 | 0.00377544 | |
| cg04523589 | 0.00684204 | −0.7146839 | 8.68E−06 | 0.00382695 | |
| cg03014680 | 0.04644373 | −0.50304455 | 9.17E−06 | 0.00397168 | |
| cg23064554 | 0.01165343 | −0.65791936 | 9.52E−06 | 0.00405678 | |
| cg00596686 | 0.00054774 | −1.10548141 | 1.00E−05 | 0.00419787 | |
| cg07374928 | 0.14938568 | 0.34354429 | 1.16E−05 | 0.0047815 | |
| cg03580247 | 0.21489245 | −0.32745239 | 1.21E−05 | 0.00492063 | |
| cg06394229 | 0.00151953 | −0.84874286 | 1.45E−05 | 0.00579498 | |
| cg22543648 | 0.04378941 | −0.53224002 | 1.66E−05 | 0.00651281 | |
| cg05154390 | 0.0657746 | −0.47829094 | 1.81E−05 | 0.00698784 | |
| cg12818699 | 0.00233247 | −0.78112404 | 1.89E−05 | 0.00718256 | |
| cg05869585 | 0.00013 | −1.15359375 | 2.23E−05 | 0.00835274 | |
| cg11136251 | 0.02454302 | 0.63765012 | 2.27E−05 | 0.00840484 | |
| cg09914444 | 0.05005868 | −0.54118725 | 2.38E−05 | 0.00866615 | |

### REFERENCES

1. Busse W W, Morgan W J, Gergen P J, et al. Randomized trial of omalizumab (anti-IgE) for asthma in inner-city children. N Engl J Med 2011; 364:1005-15.
2. Holgate S T, Djukanovic R, Casale T, Bousquet J. Anti-immunoglobulin E treatment with omalizumab in allergic diseases: an update on anti-inflammatory activity and clinical efficacy. Clin Exp Allergy 2005; 35:408-16.
3. Sheinkopf L E, Rafi A W, Do L T, Katz R M, Klaustermeyer W B. Efficacy of omalizumab in the treatment of atopic dermatitis: a pilot study. Allergy and asthma proceedings: the official journal of regional and state allergy societies 2008; 29:530-7.
4. Kay A B. Allergy and allergic diseases. Second of two parts. N Engl J Med 2001; 344:109-13.
5. Galli S J, Tsai M. IgE and mast cells in allergic disease. Nat Med 2012; 18:693-704.
6. Moller G M, de Jong T A, van der Kwast T H, et al. Immunolocalization of interleukin-4 in eosinophils in the bronchial mucosa of atopic asthmatics. Am J Respir Cell Mol Biol 1996; 14:439-43.
7. Nouri-Aria K T, O'Brien F, Noble W, Jabcobson M R, Rajakulasingam K, Durham S R. Cytokine expression during allergen-induced late nasal responses: IL-4 and IL-5 mRNA is expressed early (at 6 h) predominantly by eosinophils. Clin Exp Allergy 2000; 30:1709-16.
8. Weidinger S, Gieger C, Rodriguez E, et al. Genome-wide scan on total serum IgE levels identifies FCER1A as novel susceptibility locus. PLoS Genet 2008; 4:e1000166.
9. Moffatt M F, Gut I G, Demenais F, et al. A large-scale, consortium-based genomewide association study of asthma. N Engl J Med 2010; 363:1211-21.
10. Granada M, Wik J B, Tuzova M, et al. A genome-wide association study of plasma total IgE concentrations in the Framingham Heart Study. J Allergy Clin Immunol 2012; 129:840-5 e21.
11. Bird A. DNA methylation patterns and epigenetic memory. Genes Dev 2002; 16:6-21.
12. Bird A P. CpG islands as gene markers in vertebrate nucleus. Trends Genet 1987; 3:342-7.
13. Feinberg A P. Phenotypic plasticity and the epigenetics of human disease. Nature 2007; 447:433-40.
14. Lohning M, Richter A, Radbruch A. Cytokine memory of T helper lymphocytes. Advances in immunology 2002; 80:115-81.
15. Irizarry R A, Ladd-Acosta C, Carvalho B, et al. Comprehensive high-throughput arrays for relative methylation (CHARM). Genome Res 2008; 18:780-90.
16. Reinius L E, Acevedo N, Joerink M, et al. Differential DNA methylation in purified human blood cells: implications for cell lineage and studies on disease susceptibility. PLoS ONE 2012; 7:e41361.
17. Dixon A L, Liang L, Moffatt M F, et al. A genome-wide association study of global gene expression. Nat Genet 2007; 39:1202-7.
18. Moffatt M F, Kabesch M, Liang L, et al. Genetic variants regulating ORMDL3 expression contribute to the risk of childhood asthma. Nature 2007; 448:470-3.
19. Definition and classification of chronic bronchitis for clinical and epidemiological purposes. A report to the Medical Research Council by their Committee on the Aetiology of Chronic Bronchitis. Lancet 1965; 1:775-9.
20. Standards for the diagnosis and care of patients with chronic obstructive pulmonary disease (COPD) and asthma. This official statement of the American Thoracic Society was adopted by the ATS Board of Directors, November 1986. The American review of respiratory disease 1987; 136:225-44.
21. Begin P, Tremblay K, Daley D, et al. Association of urokinase-type plasminogen activator with asthma and atopy. Am J Respir Crit Care Med 2007; 175:1109-16.
22. Daniels S E, Bhattacharrya S, James A, et al. A genome-wide search for quantitative trait loci underlying asthma. Nature 1996; 383:247-50.
23. Ferland C, Guilbert M, Davoine F, Flamand N, Chakir J, Laviolette M. Eotaxin promotes eosinophil transmigration via the activation of the plasminogen-plasmin system. J Leukoc Biol 2001; 69:772-8.
24. Guilbert M, Ferland C, Bosse M, Flamand N, Lavigne S, Laviolette M. 5-Oxo-6,8,11,14-eicosatetraenoic acid induces important eosinophil transmigration through basement membrane components: comparison of normal and asthmatic eosinophils. Am J Respir Cell Mol Biol 1999; 21:97-104.
25. Eckhardt F, Lewin J, Cortese R, et al. DNA methylation profiling of human chromosomes 6, 20 and 22. Nat Genet 2006; 38:1378-85.
26. Liu J, Morgan M, Hutchison K, Calhoun V D. A study of the influence of sex on genome wide methylation. PLoS One; 5:e10028.
27. Christensen B C, Houseman E A, Marsit C J, et al. Aging and environmental exposures alter tissue-specific DNA methylation dependent upon CpG island context. PLoS Genet 2009; 5:e1000602.
28. Boks M P, Derks E M, Weisenberger D J, et al. The relationship of DNA methylation with age, gender and genotype in twins and healthy controls. PLoS One 2009; 4:e6767.
29. Heijmans B T, Kremer D, Tobi E W, Boomsma D I, Slagboom P E. Heritable rather than age-related environmental and stochastic factors dominate variation in DNA methylation of the human IGF2/H19 locus. Hum Mol Genet 2007; 16:547-54.
30. Reik W. Stability and flexibility of epigenetic gene regulation in mammalian development. Nature 2007; 447:425-32.
31. Deaton A M, Webb S, Kerr A R, et al. Cell type-specific DNA methylation at intragenic CpG islands in the immune system. Genome Res 2011; 21:1074-86.
32. Calvanese V, Fernandez A F, Urdinguio R G, et al. A promoter DNA demethylation landscape of human hematopoietic differentiation. Nucleic Acids Res 2012; 40:116-31.
33. Kita H. Eosinophils: multifaceted biological properties and roles in health and disease. Immunol Rev 2011; 242:161-77.
34. Rothenberg M E, Owen W F, Jr., Silberstein D S, et al. Human eosinophils have prolonged survival, enhanced functional properties, and become hypodense when exposed to human interleukin 3. J Clin Invest 1988; 81:1986-92.
35. Haldar P, Pavord I D, Shaw D E, et al. Cluster analysis and clinical asthma phenotypes. Am J Respir Crit Care Med 2008; 178:218-24.
36. Pavord I D, Korn S, Howarth P, et al. Mepolizumab for severe eosinophilic asthma (DREAM): a multicentre, double-blind, placebo-controlled trial. Lancet 2012; 380:651-9.
37. Tykocinski L O, Hajkova P, Chang H D, et al. A critical control element for interleukin-4 memory expression in T helper lymphocytes. J Biol Chem 2005; 280:28177-85.
38. Ansel K M, Djuretic I, Tanasa B, Rao A. Regulation of Th2 differentiation and Il4 locus accessibility. Annu Rev Immunol 2006; 24:607-56.
39. Sanderson C J. Interleukin-5, eosinophils, and disease. Blood 1992; 79:3101-9.
40. Lopez A F, Sanderson C J, Gamble J R, Campbell H D, Young I G, Vadas M A. Recombinant human interleukin 5 is a selective activator of human eosinophil function. The Journal of experimental medicine 1988; 167:219-24.
41. Shindou H, Hishikawa D, Nakanishi H, et al. A single enzyme catalyzes both platelet-activating factor production and membrane biogenesis of inflammatory cells. Cloning and characterization of acetyl-CoA:LYSO-PAF acetyltransferase. J Biol Chem 2007; 282:6532-9.
42. Kruse S, Mao X Q, Heinzmann A, et al. The Ile198Thr and Ala379Val variants of plasmatic PAF-acetylhydrolase impair catalytical activities and are associated with atopy and asthma. Am J Hum Genet 2000; 66:1522-30.
43. Peachman K K, Lyles D S, Bass D A. Mitochondria in eosinophils: functional role in apoptosis but not respiration. Proc Natl Acad Sci USA 2001; 98:1717-22.
44. Chu V T, Frohlich A, Steinhauser G, et al. Eosinophils are required for the maintenance of plasma cells in the bone marrow. Nature immunology 2011; 12:151-9.
45. Breitling L P, Yang R, Korn B, Burwinkel B, Brenner H. Tobacco-smoking-related differential DNA methylation: 27K discovery and replication. Am J Hum Genet 2011; 88:450-7.
46. Wan E S, Qiu W, Baccarelli A, et al. Cigarette smoking behaviors and time since quitting are associated with differential DNA methylation across the human genome. Hum Mol Genet 2012.
47. Roessler J, Ammerpohl O, Gutwein J, et al. Quantitative cross-validation and content analysis of the 450k DNA methylation array from Illumina, Inc. BMC research notes 2012; 5:210.
48. M. Niedzwiecki, H. Zhu, L. Corson et al. Prenatal exposure to allergen, DNA methylation, and allergy in grandoffspring mice. Allergy 2012; 67(6): 904-910.
49. Relton C L, Davey Smith G. Two-step epigenetic Mendelian randomization: a strategy for establishing the causal role of epigenetic processes in pathways to disease. International journal of epidemiology. 2012; 41(1):161-176.
50. Willer C J, Li Y, Abecasis G R. METAL: fast and efficient meta-analysis of genomewide association scans. Bioinformatics. 2010; 26(17):2190-2191.
51. Howie B, Fuchsberger C, Stephens M, Marchini K, Abecasis G R. Fast and accurate genotype imputation in genome-wide association studies through pre-phasing. Nat Genet. 2012; 44(8):9550959.
52. Lawlor D A, Harbord R M, Sterne J A, Timpson N, Davey Smith G, Mendelian randomization: using genes as instruments for making causal inferences in epidemiology. Stat Med. 2008; 27(8):1133-1163.
53. Touleimat N, Tost J. Complete pipeline for Infinium ((R)) Human Methylation 450K BeadCHip data processing using subset quantile normalization for accurate DNA methylation estimation. Epigenomics. 2012; 4(3):325-341
54. Du P, Kibbe W A, Lin S M. lumi: a pipeline for processing Illumina microarray. Bioinformatics. 2008; 24(13):1547-1548
55. Houseman E A, et al. DNA methylation arrays as surrogate measures of cell mixture distribution. BMC Bioinformatics. 2012; 13:86.

We claim:

1. A method of identifying an eosinophil IgE mediated allergic inflammation in a human subject, comprising the steps of:
i) extracting DNA from a blood sample obtained from said human subject,
ii) detecting in said DNA the level of methylation in one or more promoter regions associated with one or more genes selected from the group consisting of LPCAT2, IL5RA, ZNF22, L2HGDH, IL4, SLC25A33, RB1, SERPINC1, TFF1, SKC17A4, L2HGDH, TMEM86B, COL15A1, CEL, SPINK4, ADARB1, SEPT12, TMEM52B, FAM112A, SLC7A11, KEL, PIK3CB, TMEM41A, PDE6H, KLF1, ITAG2B, PRG3, SLMAP, PRG2, EFNA3, SLC43A3, CLC, ALDH3B2, GATA1, CCR3 and IL1RL1 by a method selected from methylation specific PCR, Hpall tiny fragment enriched by ligation-mediated PCR assay, ChIP-on-chip, restriction landmark genomic scanning, methylated DNA immune precipitation, pyrosequencing, methylation bead array analysis, microarray analysis, bisulfate sequencing and methylCpG binding proteins;
(iii) designating levels of methylation from step ii) in the one or more promoter regions as low methylation, wherein it is at least 2 standard deviations less than the mean level of methylation in the same one or more promoter regions in a control sample, and
(iv) assigning the subject as a member of the patient population with eosinophil mediated allergic inflammation where there is low methylation in one or more of the promoter regions.

2. (canceled)

3. A method according to claim 1, wherein the low methylation is defined as a level of methylation that is below the level of methylation for the 95^{th} percentile of a control population.

4. A method according to claim 1, wherein the one or more promoter regions are associated with LPCAT2 and one or more genes selected from the group consisting of IL5RA, ZNF22, L2HGDH, IL4, SLC25A33, RB1, SERPINC1, TFF1, SKC17A4, L2HGDH, TMEM86B, COL15A1, CEL, SPINK4, ADARB1, SEPT12, TMEM52B, FAM112A, SLC7A11, KEL, PIK3CB, TMEM41A, PDE6H, KLF1, ITAG2B, PRG3, SLMAP, PRG2, EFNA3, SLC43A3, CLC, ALDH3B2, GATA1, CCR3 and IL1RL1.

5. A method according to claim 1, wherein the one or more promoter regions is associated with a gene selected from the group consisting of SLC25A33, LPCAT2, L2HGDH and a combination thereof.

6. (canceled)

7. A method according to claim 1, wherein the one or more promoter regions is associated with a gene selected from the group consisting of CEL, CLC and a combination thereof.

8. A method according to claim 1, wherein the one or more promoter regions is associated with a gene selected from the group consisting of ZNF22, RB1, KLF and a combination thereof.

9. A method according to claim 1, wherein the one or more promoter regions is associated with a gene selected from the group consisting of PRG3, SERPINC1, TFF1, SPINK and a combination thereof.

10. A method according to claim 1, wherein promoter regions associated with each of the genes LPCAT2, IL5RA, ZNF22, L2HGDH, IL4, SLC25A33, RB1, SERPINC1, TFF1, SKC17A4, L2HGDH, TMEM86B, COL15A1, CEL, SPINK4, ADARB1, SEPT12, TMEM52B, FAM112A, SLC7A11, KEL, PIK3CB, TMEM41A, PDE6H, KLF1, ITAG2B, PRG3, SLMAP, PRG2, EFNA3, SLC43A3, CLC, ALDH3B2, GATA1, CCR3 and IL1RL1 is evaluated.

11. A method according to claim 1, wherein the number of genes analysed is 1, 2, 3, 4, 5, 6, 7, 8, 9 or 10.

12. A method according to claim 1, wherein the eosinophil IgE mediated allergic inflammation is manifest in the subject as asthma, rhinitis, seasonal rhinitis, atopic dermatitis, anaphylaxis or a combination thereof, such as atopic asthma.

13. A method according to claim 1, which further comprises a first step of determining if the subject suffers from an allergic inflammatory condition, such as asthma, rhinitis, seasonal rhinitis, atopic dermatitis, anaphylaxis or a combination thereof, such as atopic asthma.

14. A method according to claim 1, which further comprises the step of administering to a subject assigned as a member of the patient population with eosinophil IgE mediated inflammation a therapeutically effective amount of a medication for said eosinophil IgE mediated inflammation.

15. (canceled)

16. A method according to claim 14, wherein the medicament is an antibody or binding fragment thereof.

17. A method according to claim 14, wherein the medicament is an inhibitor of IL-5 or IL-5 receptor, an inhibitor of IL-13 or IL-13 receptor, an inhibitor of IgE or an inhibitor of M1 prime.

18. A method according to claim 17, wherein the inhibitor is selected from the group consisting of Benralizumab, Mepolizumab, Reslizumab, Tralokinumab, Lebrikizumab, Omalizumab, Quilizumab and a combination thereof.

19. A method according to claim 14, wherein the medicament increases the level of methylation in a target genomic region associated with eosinophil IgE mediated inflammation.

20. A method according to claim 14, further comprising the step of administering a known therapy.

21. A method according to claim 20, wherein the known therapy is a therapy directed towards eosinophils, such as steroid therapy, beta2 agonists and biological therapeutic agents, for example an antibody or binding fragment thereof.

22. A method according to claim 1, wherein the DNA sample is obtained from eosinophils.

23. (canceled)

24. A method of detecting the level of methylation in one or more promoter regions associated with one or more genes selected from the group consisting of LPCAT2, IL5RA, ZNF22, L2HGDH, IL4, SLC25A33, RB1, SERPINC1, TFF1, SKC17A4, L2HGDH, TMEM86B, COL15A1, CEL, SPINK4, ADARB1, SEPT12, TMEM52B, FAM112A, SLC7A11, KEL, PIK3CB, TMEM41A, PDE6H, KLF1, ITAG2B, PRG3, SLMAP, PRG2, EFNA3, SLC43A3, CLC, ALDH3B2, GATA1, CCR3 and IL1RL1, comprising the steps of:
i) extracting DNA from a blood sample obtained from said human subject, and
ii) detecting the level of methylation in one or more promoter regions associated with one or more genes selected from the group consisting of LPCAT2, IL5RA, ZNF22, L2HGDH, IL4, SLC25A33, RB1, SERPINC1, TFF1, SKC17A4, L2HGDH, TMEM86B, COL15A1, CEL, SPINK4, ADARB1, SEPT12, TMEM52B, FAM112A, SLC7A11, KEL, PIK3CB, TMEM41A, PDE6H, KLF1, ITAG2B, PRG3, SLMAP, PRG2, EFNA3, SLC43A3, CLC, ALDH3B2, GATA1, CCR3 and IL1RL1 by conducting methylation specific PCR, Hpall tiny fragment enriched by ligation-mediated PCR assay, ChIP-on-chip, restriction landmark genomic scanning, methylated DNA immune precipitation, pyrosequencing, methylation bead array analysis, microarray analysis, bisulfite sequencing or methylCpG binding proteins analysis.
